SEQUENCE LISTING (23 sequences)

SEQ ID NO: 1 - DNA, 31 bp, Homo sapien
ggtgtggcagaagcaccaggaaagacaaaag

SEQ ID NO: 2 - DNA, 31 bp, Homo sapien
ggagggagtggtgcagggtgtggcaacagtg

SEQ ID NO: 3 - DNA, 30 bp, Homo sapien
gggagtggtgcatgatgtggcaacagtggc

SEQ ID NO: 4 - DNA, 30 bp, Homo sapien
gtggtgcatggtgtgacaacagtggctgag

SEQ ID NO: 5 - DNA, 32 bp, Homo sapien
ggctccaaaaccaagaagggagtggtgcatgg

SEQ ID NO: 6 - DNA, 51 bp, Homo sapien
aaccaaacagggtgtggcagaagcagcaggaaagacaaaagagggtgttct

SEQ ID NO: 7 - DNA, 51 bp, Homo sapien
aaccaaacagggtgtggcagaagcaccaggaaagacaaaagagggtgttct

SEQ ID NO: 8 - DNA, 51 bp, Homo sapien
ccaaaaccaaggagggagtggtgcatggtgtggcaacagtggctgagaaga

SEQ ID NO: 9 - DNA, 51 bp, Homo sapien
ccaaaaccaaggagggagtggtgcagggtgtggcaacagtggctgagaaga

SEQ ID NO: 10 - DNA, 51 bp, Homo sapien
aaaaccaaggagggagtggtgcatggtgtggcaacagtggctgagaagacc

SEQ ID NO: 11 - DNA, 51 bp, Homo sapiens
aaaaccaaggagggagtggtgcatgatgtggcaacagtggctgagaagacc

SEQ ID NO: 12 - DNA, 51 bp, Homo sapiens; identical to SEQ ID NO: 10

SEQ ID NO: 13 - DNA, 51 bp, Homo sapiens
caaggagggagtggtgcatggtgtgacaacagtggctgagaagaccaaaga

SEQ ID NO: 14 - DNA, 50 bp, Homo sapiens
gtttttgtaggctccaaaaccaaggagggagtggtgcatggtgtggctga

SEQ ID NO: 15 - DNA, 50 bp, Homo sapiens
gtttttgtaggctccaaaaccaagaagggagtggtgcatggtgtggctga

SEQ ID NO: 16 - DNA, 51 bp, Homo sapiens; identical to SEQ ID NO: 6

SEQ ID NO: 17 - DNA, 51 bp, Homo sapiens; identical to SEQ ID NO: 7

SEQ ID NO: 18 - DNA, 62 bp, Homo sapiens
taggcccaaaaccaaggagggagtggtgcatggtgtggcaacagtggctgagaagaccaaag

SEQ ID NO: 19 - DNA, 62 bp, Homo sapiens
taggcccaaaaccaaggagggagtggtgcagggtgtggcaacagtggctgagaagaccaaag

SEQ ID NO: 20 - DNA, 62 bp, Homo sapiens; identical to SEQ ID NO: 19

SEQ ID NO: 21 - DNA, 62 bp, Homo sapiens
taggcccaaaaccaaggagggagtggtgcatgatgtggcaacagtggctgagaagaccaaag

SEQ ID NO: 22 - DNA, 62 bp, Homo sapiens
taggcccaaaaccaaggagggagtggtgcatggtgtgacaacagtggctgagaagaccaaag

SEQ ID NO: 23 - DNA, 62 bp, Homo sapiens
taggcccaaaaccaagaagggagtggtgcatggtgtggcaacagtggctgagaagaccaaag

(19) United States
(12) Patent Application Publication
(10) Pub. No.: US 2022/0325346 A1
(43) Pub. Date: Oct. 13, 2022

(54) USE OF SNCA-MEDIATED GENES FOR DIAGNOSIS AND TREATMENT OF PARKINSON'S DISEASE

(71) Applicant: SELONTERRA, INC., SAN MATEO, CA (US)

(72) Inventors: ANNE URFER-BUCHWALDER, Belmont, CA (US); ROMAN URFER, Belmont, CA (US)

(73) Assignee: SELONTERRA, INC., SAN MATEO, CA (US)

(21) Appl. No.: 17/633,667

(22) PCT Filed: Jul. 9, 2020
(86) PCT No.: PCT/US20/41452

§ 371 Date: Feb. 8, 2022

Related U.S. Application Data
(60) Provisional application No. 62/871,875, filed on Jul. 9, 2019.

(51) Int. Cl. C12Q 1/6883

(52) U.S. Cl. CPC C12Q 1/6883; C12Q 2600/158

(57) ABSTRACT

The present invention provides compositions and methods using SNCA-mediated genes and expression products thereof for diagnosis, treatment and prevention of Parkinson's disease. The present invention also relates to a method of identifying therapeutic agents to treat and diagnose Parkinson's disease based on SNCA-mediated genes.

### CLAIM OF PRIORITY

This application is a National Stage Application of PCT application S/N PCT/US20/41452 filed on Jul. 9, 2020, which claims priority of U.S. provisional application 62/871,875 with filing date of Jul. 9, 2019 the entire contents of which are hereby incorporated by reference in their entirety FIELD OF THE INVENTION

The present invention relates to a method of diagnosing, preventing and treating Parkinson's disease in carriers of a SNCA genomic variant. Methods of the invention are based at least in part on modulating one or more genes, or their expression products, from a set of genes identified by the present invention as being connected to Parkinson's disease (referred to herein as the “SNCA-mediated genes”). The present invention also relates to a method of using the modulation of the activity and/or expression of SNCA-mediated genes to treat and diagnose Parkinson's disease in carriers of a SNCA genomic variant.

### BACKGROUND OF THE INVENTION

Parkinson's disease (PD) affects millions of people worldwide. Currently, however, there are no reliable and effective methods for treatment or prevention of PD. It is believed that PD is caused by a combination of environmental, age and genetic factors. PD consists of idiopathic and familial forms. The underlying neurodegeneration in PD often goes unrecognized in its very early stages where treatments might be most effective. Current Food and Drug Administration (FDA) approved Parkinson's drugs have significant side effects and modest effects on improving the patient's daily functioning but do not slow down the disease process or treat the underlying pathology.

The biochemical basis of the very tissue specific degeneration of neurons in the substantia nigra in PD is not fully understood.

Unfortunately, it is not clear whether the biochemical events observed are a cause or a result of the development of PD. The conventional research apparently does not relate to the actual mechanism of causation of PD but rather only relates to the consequences of the underlying mechanism that causes PD. If one can gain detailed knowledge of the underlying mechanism for development of PD, it would be possible to accurately diagnose, treat and/or prevent development of PD.

One tantalizing clue towards possible treatments for PD comes from genetic research which revealed that people carrying specific alleles of SNCA are at substantially increased risk of developing PD. This genetic risk of SNCA alleles extends to both the idiopathic and familial forms of PD. This genetic discovery has triggered substantial investigation into the role SNCA may play in the development of PD. To date there is no generally accepted explanation for the correlation between SNCA activity, which occurs throughout the brain, and the observed pathological effects of PD which are limited to a very specific area of the brain, the substantia nigra. Here again, this research area has been frustrating and has not yet led to new treatments for PD.

Since current treatment of PD only treats the symptoms and not the underlying cause of PD, there is still a need for a method for treating PD by treating the actual cause of PD.

### SUMMARY OF THE INVENTION

Some aspects of the present invention are based on the discovery of a set of genes that are directly responsible for the development of PD. This discovery by the present invention provides multiple new avenues for diagnosing, preventing and/or treating PD.

The human SNCA gene (ENSG00000145335; chromosomal location of SNCA 4:89724099-89838315) is located on human chromosome 4 and is defined as the “SNCA wild-type”. A “SNCA genomic variant” is a human SNCA gene that has one or more of the nucleotides present in SNCA wild-type substituted by one or more different nucleotides. In some instances, the nucleotide change results in an amino acid change in the encoded SNCA protein. In other instances, the nucleotide change does not affect the resulting amino acid sequence of the encoded SNCA protein. There are many naturally occurring human SNCA genomic variants known to the skilled in the art. For instance, rs104893878, rs201106962, rs431905511, rs104893877, rs104893875, rs356182, rs2736990, rs2572324, rs7684318, rs894278, rs76642636 and rs764216470 are examples of genomic variants in the human SNCA gene. Rs numbers are accession numbers used by the ones skilled in the art and by databases to refer to specific single nucleotide polymorphisms (SNP) in the human genome.

Variants in the human SNCA gene have been identified in numerous families with heritable Parkinson's disease. SNCA genomic variants that segregate with PD in large familiesⁱ include rs104893878 (A30P), rs201106962 (H50Q), rs431905511 (G51D) and rs104893877 (A53T). SNCA genomic variants have also been associated with idiopathic cases of PDⁱⁱ and include rs356182, rs2736990, rs2572324, rs7684318, and rs894278.

The present invention recognized that changes in the protein sequence of SNCA do not explain important aspects of the link of SNCA genomic variants to PD. While the disease manifests itself in a degeneration of the substantia nigra, the expression of the SNCA protein occurs in neurons throughout the brain.

Consistent with the observed expression pattern, animals in which the SNCA wild-type and SNCA genomic variants were overexpressed using transgenic animal technology or adeno-associated virus mediated expression did not display consistent specific nigrostriatal degeneration. Further, while AAV-mediated expression of SNCA yielded dopaminergic neurodegeneration there was no significant difference between animals expressing the SNCA wild-type and SNCA genomic variants, respectively.

A common PD-associated risk variant was identified in a non-coding distal enhancer element in intron 4 of the SNCA gene that regulates the expression of SNCA itselfⁱⁱⁱ. The SNCA genomic variants rs356166 and rs356168 are associated with PD disease risk and protection, respectively. The transcriptional dysregulation of SNCA was associated with sequence-dependent binding of the brain-specific transcription factors EMX2 and NKX6-1 to this locus. The scope of that study was limited to the analysis of the rs3561666 and rs356168 loci located in intron 4 of the SNCA gene and its effects on SNCA expression. The authors of that study did not disclose effects of SNCA genomic variants on gene expression other than SNCA (i.e, SNCA-mediated genes). The present invention identified transcription factor binding sites in SNCA genomic variants and discloses unexpected effects in the transcription of genes (i.e., SNCA-mediated genes) in the chromosomal vicinity of SNCA (i.e. adjacent genes on the same chromosome or genes encoded on a separate chromosome held in close spatial proximity), excluding SNCA itself.

One particular aspect of the invention is based on the analysis of the gene structure of SNCA genomic variants including SNCA genomic variants located in exons, the protein-encoding section of the SNCA gene, and the unexpected discovery that these SNCA genomic variants act at their DNA level, create de novo recognition motifs for transcription factors ID4, STAT5a::STAT5b, TCF3/4, KLF1/4/5/12, TBX4/5, MAFG::NE2FL1, SP3, PAX2, MGA, SREBF2, FOXD2, FOXO4/FOXO6, MEIS1 and MEIS3, or remove recognition motifs for CEBPA and NFIX. This finding by the present invention reveals that SNCA genomic variant alleles are unexpectedly modulating (e.g., activating or suppressing) transcription of a range of genes besides of SNCA itself. These genes, located in the chromosomal vicinity of SNCA, excluding SNCA, are herein referred to as SNCA-mediated genes. SNCA-mediated genes are located on human chromosome 4 where transcription factors act in cis, and on human chromosome 12 where transcription factors act in trans. These genes are not activated (or suppressed) to the same extent by the SNCA wild-type, thereby providing an explanation for the correlation of SNCA genomic variants with PD in carriers of a SNCA genomic variant.

As discussed herein, SNCA-mediated gene expression can result in inhibition or activation of a particular SNCA-mediated gene or the activity of the gene expression product. Accordingly, throughout this disclosure, it should be understood that when the term “modulate” is used in reference to SNCA-mediated gene expression or the gene expression product thereof or the activity of the gene expression product thereof, the term “modulate” can be substituted with the term “increase”, “activate”, “decrease”, “suppress”, “inhibit” or similar terms. “Gene expression” is used to refer to the creation of an mRNA and “gene expression product” can refer to the mRNA or a protein translated from the mRNA. For example, if a particular gene expression (or the activity of the gene expression product thereof) is reduced or suppressed due to the presence of a SNCA genomic variant, then the methods of the invention will be directed to increasing the gene expression (or the activity of the gene expression product thereof). Similarly, if a particular gene expression (or the activity of the gene expression product thereof) is increased due to the presence of a SNCA genomic variant, then the methods of the invention will be directed to inhibiting the gene expression (or the activity of the gene expression product thereof). It will be understood by those of skill in the art that increasing or decreasing gene expression can directly increase or decrease the activity of a gene expression product by increasing or decreasing the amount of the gene expression product. The activity of gene expression products can alternatively be increased or decreased by, for example, a molecule that interacts with a gene expression product that is an mRNA or a protein. Alternatively, gene expression activity or a gene expression product can be increased or decreased by a transcription factor or a molecule that interacts with a transcription factor. Additional methods of modulating expression are known and the scope of the invention is not meant to be limited by the examples provided herein.

The term “modulate” when used in reference to a SNCA-mediated gene expression includes reducing or increasing transcription and/or translation of the SNCA-mediated gene. This can include downregulating or complete suppression or upregulating of the expression products of a SNCA-mediated gene as compared to a control (e.g., in the absence of a molecule). The term “modulate” when used in reference to the activity of a SNCA-mediated gene expression product includes reducing or increasing the activity of the SNCA-mediated gene. Typically, methods of the invention show at least about 25%, typically at least about 50%, and often at least about 75% modulation of SNCA-mediated gene expression or activity of the expression product thereof. In reference to a gene expression product, “modulate” can also refer to causing a change in the activity of the gene expression product itself.

An aspect of the invention includes a method for detecting a change in the expression of at least one SNCA-mediated gene comprising isolating total RNA from a tissue or cell sample obtained from a carrier of a SNCA genomic variant suspected of having Parkinson's disease. Embodiments of this aspect include wherein the tissue or cell sample is whole blood, blood plasma, blood serum, sputum, saliva, urine, lymph or cerebrospinal fluid, human homozygous or heterozygous SNCA genomic variant carrying neurons, oligodendrocytes, buccal cells, or skin fibroblasts. Embodiments include assays including cell-based assay for detecting a change in the expression of, or an activity of a gene expression product encoded by, a SNCA-mediated gene. In certain embodiments the cells are neuronal cells, neuronal progenitor cells, differentiated neurons or oligodendrocytes. In particular embodiments the cells are human cells.

The method includes synthesizing and amplifying SNCA-mediated gene cDNA from said tissue or cell sample and identifying a change in the expression of at least one SNCA-mediated gene, wherein the change in the expression of the at least one SNCA-mediated gene comprises a change in the amount of mRNA encoded by the at least one SNCA-mediated gene compared to the amount in total RNA isolated from a same tissue or cell sample from a healthy subject or a subject homozygous for a wild-type SNCA gene. Embodiments of this aspect include wherein the expression of at least one SNCA-mediated gene is greater than its expression in total RNA isolated from the same tissue or cell sample obtained from a healthy subject homozygous for a wild-type SNCA gene. Embodiments also include methods wherein the expression of the least one SNCA-mediated gene is less than the expression of the SNCA-mediated gene in total RNA isolated from the same tissue or cell sample obtained from a healthy subject or a subject homozygous for a wild-type SNCA gene. Embodiments also include methods wherein the change in the expression of the at least one SNCA-mediated gene is detected in total RNA isolated from tissue or cell samples obtained from carriers of a SNCA genomic variant with PD.

An embodiment of the invention includes a method for detecting a change in the expression of, or an activity of a gene expression product encoded by, a SNCA-mediated gene comprising measuring the expression of, or the activity of a gene expression product encoded by, a SNCA-mediated gene in cells carrying a SNCA genomic variant and detecting a change in the expression of or an activity in response to a molecule, wherein the change detected comprises a change in the synthesis of a gene expression product, an activity of the gene expression product or the expression of an mRNA encoded by the SNCA-mediated gene. Further embodiments include conducting the method in a cell based assay. In certain embodiments the cells are neuronal cells, neuronal progenitor cells, differentiated neurons or oligodendrocytes. In particular embodiments the cells are human cells. Embodiments include methods wherein the expression of, or an activity of a gene expression product encoded by, the SNCA-mediated gene increases or decreases in response to a molecule. In particular embodiments, the method detects the modulation in expression of, or the activity of a gene expression product encoded by, the SNCA-mediated gene by a change in the amount of phospho-Ser⁴⁷³-Akt, total Akt, or a combination thereof. In a further embodiment, the method detects the modulation in expression of, or the activity of a gene expression product encoded by, the SNCA-mediated gene by a change in the amount of Notch. In particular embodiments the molecule is selected from small molecules, oligonucleotides (including short interfering RNAs, RNAs, long non-coding RNAs and aptamers), peptides, polypeptides (including aptamers, zinc fingers and fragments thereof), proteins (including antibodies and fragments thereof) as well as derivatives or modified forms thereof. In certain embodiments the molecule is potentially useful for treating or preventing the progression of PD in a carrier of a SNCA genomic variant.

One particular aspect of the invention provides a method of modulating (i.e., increasing or decreasing) the SNCA-mediated (i.e., regulated) expression of a gene or the activity of gene product thereof. In some embodiments, modulation of the SNCA genomic variant regulated gene expression is achieved by contacting a cell that is capable of or is expressing a gene mediated by the SNCA genomic variant with a molecule. As used herein unless the context requires otherwise, the terms “molecule” and “compound” are used interchangeably herein and refers to any molecule known to one skilled in the art, such as, but not limited to, small molecules, oligonucleotides (including short interfering RNAs and aptamers), peptides, polypeptides (including aptamers, zinc fingers and fragments thereof), proteins (including antibodies and fragments thereof) as well as derivatives or modified forms thereof. In some embodiments, a molecule is delivered using a viral vector. In some embodiments, the cell is a neuronal cell, a neuronal progenitor cell, a differentiated neuron, an oligodendrocyte, a fibroblast or a lymphocyte. In other embodiments, the modulation of the activity of a SNCA-mediated gene expression product is achieved by contacting said gene expression product with a molecule that is capable of selectively modulating the activity of said gene expression product.

In aspects of the invention, SNCA-mediated genes are located on human chromosome 4 within about 2 Mb upstream or downstream of the location of SNCA (ENSG00000145335; chromosomal location of SNCA 4:89724099-89838315). In certain embodiments of this aspect, the SNCA-mediated gene is at least one gene selected from the group comprising of KLHL8, PTPN13, HSD17B13, HSD17B11, NUDT9, SPARCL1, DMP1, IBSP, MEPE, SPP1, PKD2, ABCG2, PPM1K, HERC6, HERC5, PIGY, PYURF, HERC3, NAP1L5, FAM13A, TIGD2, GPRIN3, SNCA-AS1, MMRN1, CCSER1, GRID2, and a combination thereof.

In another aspect of the invention, SNCA-mediated genes are located on human chromosome 12. In a certain embodiment of this aspect, the SNCA-mediated gene is PDZRN4.

In a further embodiment, the SNCA-mediated gene is at least one gene from the group comprising ABCG2, PPM1K, HERC3, HERC5, HERC6, SPARCL1, MMRN1, PDZRN4 or a combination thereof. In yet a further embodiment, the SNCA-mediated gene is at least one gene from the group comprising HERC5, HERC6, SPARCL1, MMRN1, PDZRN4, or a combination thereof.

In one particular embodiment, the expression or activity of a SNCA-mediated gene (or gene expression product thereof) is modulated using a molecule or a compound. As used herein, the terms “SNCA-mediated gene,” “gene mediated by SNCA genomic variant” and the like are used interchangeably herein. In some embodiments SNCA-mediated genes are in a cis relationship to SNCA, located on human chromosome 4 within about 5 Mb, typically within about 4 Mb, often within about 3 Mb, and most often within about 2 Mb upstream or downstream of the location of SNCA (ENSG00000145335; chromosomal location of SNCA 4:89724099-89838315) or gene(s) located on human chromosome 12.

For additional clarity, an SNCA-mediated gene is one with sufficient (intra or inter-chromosomal proximity to the SNCA gene that its expression level is modified by transcription factors which bind to transcription factor binding sites in the genomic (e.g. exon or intron) sequence of the SNCA gene. Such SNCA-mediated genes have a standard expression level based on the wild-type (non-disease causing) allele. An allele with a SNCA genomic variant, such as those represented by the single nucleotide polymorphisms (SNPs) disclosed herein, with modified transcription factor binding sites, have significantly different transcription and/or expression levels of SNCA-mediated genes, thereby putting the carrier of a SNCA genomic variant at risk of PD.

The terms “about”, “approximately”, and the like when used to describe a numeric value, are used interchangeably herein and are not intended to limit the scope of the invention unless indicated otherwise. The terms “about” and “approximately” refer to being within an acceptable error range for the particular value as determined by one of ordinary skill in the art, which will depend in part on how the value is measured or determined, i.e., the limitations of the measurement system, i.e., the degree of precision required for a particular purpose. For example, the terms “about” and “approximately” can mean within 1 or more than 1 standard deviation, per the practice in the art. Alternatively, the terms “about” and “approximately” when referring to a numerical value can mean±20%, typically ±10%, often ±5% and more often ±1% of the numerical value. In general, however, where particular values are described in the application and claims, unless otherwise stated, the terms “about” and “approximately” mean within an acceptable error range for the particular value.

An aspect of the invention is a method that can be used inter alia to identify a lead candidate for a drug development for treatment of PD, in a carrier of a SNCA genomic variant.

Certain embodiments of this aspect include a molecule used for modulating SNCA-mediated expression of a gene. In these embodiments, the “molecule” includes, but is not limited to, small molecules, oligonucleotides (including short interfering RNAs, RNAs and aptamers), peptides, polypeptides (including aptamers, zinc fingers and fragments thereof), proteins (including antibodies and fragments thereof) as well as derivatives or modified forms thereof.

In some embodiments of the invention an oligonucleotide is employed to modulate the expression of SNCA-mediated genes. In certain embodiments, the oligonucleotide is 11 to 30 nucleotides in length comprising consecutive nucleotide sequences within SEQ ID NO:1, SEQ ID NO:2, SEQ ID NO:3, SEQ ID NO:4 or SEQ ID NO:5. In some cases, the oligonucleotide is a single-stranded oligonucleotide while in other instances the oligonucleotide is a double-stranded oligonucleotide. The oligonucleotide can also be a phosphorothioate oligonucleotide, a phosphoramidite oligonucleotide, a methylphosphonate oligonucleotide, a locked-nucleic acid-modified oligonucleotide, a peptide nucleic acid oligonucleotide, or combinations thereof.

The oligonucleotide sequence of SEQ ID NO:1 is GGTGTGGCAGAAGCACCAGGAAAGACAAAAG corresponding to SNCA genomic variant rs104893878 (A30P). The oligonucleotide sequence of SEQ ID NO:2 is GGAGGGAGTGGTGCAGGGTGTGGCAACAGTG corresponding to SNCA genomic variant rs201106962 (H50Q). The oligonucleotide sequence of SEQ ID NO:3 is GGGAGTGGTGCATGATGTGGCAACAGTGGC corresponding to SNCA genomic variant rs431905511 (G51D). The oligonucleotide sequence of SEQ ID NO:4 is GTGGTGCATGGTGTGACAACAGTGGCTGAG corresponding to SNCA genomic variant rs104893877 (A53T). The oligonucleotide sequence of SEQ ID NO:5 is GGCTCCAAAACCAAGAAGGGAGTGGTGCATGG corresponding to SNCA genomic variant rs104893875 (E46K).

In some embodiments, the molecule further comprises a pharmaceutically acceptable carrier.

In some embodiments, the molecule is delivered by a viral vector.

Another aspect of the invention provides a method for identifying a molecule that can modulate the binding of a transcription factor to the SNCA genomic variant. This method can be used inter alia to identify a lead candidate for a drug development for treatment of PD in a carrier of a SNCA genomic variant.

An aspect of the invention provides a method for improving at least one symptom in a carrier of a SNCA genomic variant with PD comprising administering a molecule to said carrier exhibiting a change in the expression of, or the activity of a gene expression product of at least one SNCA-mediated gene, said molecule modulating a change in the expression of, or an activity of a gene expression product by, at least one SNCA-mediated gene, and said modulation correlating with the improvement of at least one symptom.

Another aspect of the invention provides a method for modulating (i.e., increase or decrease) the expression or activity of a SNCA-mediated gene or expression products thereof in a cell. The method comprises contacting a cell that is expressing a SNCA-mediated gene with a molecule that is capable of modulating the expression or activity of said gene. This method can be used inter alia to identify a lead candidate for a drug development for treatment of PD in a carrier of a SNCA genomic variant. In further embodiments, the molecule includes, but is not limited to, small molecules, oligonucleotides (including short interfering RNAs, RNAs and aptamers), peptides, polypeptides (including aptamers, zinc fingers and fragments thereof), proteins (including antibodies and fragments thereof) as well as derivatives or modified forms thereof.

Another aspect of the invention provides a method for treating a subject suffering from PD who is a carrier of a SNCA genomic variant. The method includes determining the presence of a SNCA genomic variant in the subject; and (a) if said subject carries a SNCA genomic variant, administering to said subject a molecule that is capable of modulating the expression or activity of a SNCA-mediated gene; or (b) if said subject does not carry a SNCA genomic variant, administering said subject with a molecule that is different from said molecule of (a). In some embodiments, the method can include a step of determining whether said subject is homozygous or heterozygous for the SNCA genomic variant. As used herein, the term “treating” or “treatment” or the like of a disease includes: (1) preventing the disease, i.e., causing the clinical symptoms of the disease not to develop in a mammal that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

A method of this embodiment results in improving at least one symptom in a carrier of a SNCA genomic variant with PD. The method includes administering a molecule to said carrier exhibiting a change in the expression of, or the activity of a gene expression product of at least one SNCA-mediated gene. The molecule modulates a change in the expression of, or an activity of a gene expression product of, at least one SNCA-mediated gene, and the modulation correlates with the improvement of at least one symptom. In certain aspects the modulation is an increase or decrease in the expression of, or an activity of a gene expression product encoded by, the at least one SNCA-mediated gene. In certain embodiments the expression of, or an activity of a gene expression product, of the at least one SNCA-mediated gene is modulated by administering to said carrier a molecule, said molecule causing a decrease in the expression of, or an activity of a gene expression product of a SNCA-mediated gene, and said decrease in expression or activity correlating with an improvement of at least one symptom. In another embodiment, the expression of, or an activity of a gene expression product, of the at least one SNCA-mediated gene is modulated by administering to said carrier a molecule and the molecule causing an increase in the expression of, or an activity of a gene expression product of a SNCA-mediated gene, and the increase in expression or activity correlates with an improvement of at least one symptom. In certain embodiments, the molecule comprises at least one selected from small molecules, oligonucleotides (including short interfering RNAs, RNAs, long non-coding RNAs and aptamers), peptides, polypeptides (including aptamers, zinc fingers and fragments thereof), proteins (including antibodies and fragments thereof) as well as derivatives or modified forms thereof.

An aspect of the invention is a kit for detecting a change in the expression of at least one SNCA-mediated gene in total RNA isolated from a sample obtained from a carrier of a SNCA genomic variant suspected of having PD. The kit can include an agent for detecting mRNA encoded by at least one SNCA-mediated gene comprising at least one of KLHL8, PTPN13, HSD17B13, HSD17B11, NUDT9, SPARCL1, DMP1, IBSP, MEPE, SPP1, PKD2, ABCG2, PPM1K, HERC6, HERC5, PIGY, PYURF, HERC3, NAP1L5, FAM13A, TIGD2, GPRIN3, SNCA-AS1, MMRN1, CCSER1, GRID2 and PDZRN4. In particular embodiments the kit can be used for conducting a cell based assay. In certain embodiments the cells are neuronal cells, neuronal progenitor cells, differentiated neurons or oligodendrocytes. In particular embodiments the cells are human cells. In certain embodiments the kit can be used to test molecules such as small molecules, oligonucleotides (including short interfering RNAs, RNAs, long non-coding RNAs and aptamers), peptides, polypeptides (including aptamers, zinc fingers and fragments thereof), proteins (including antibodies and fragments thereof) as well as derivatives or modified forms thereof.

The advantages of the present invention include a transformative approach to treatment of carriers of a SNCA genomic variant for PD, targeted therapies addressing dysfunction caused by SNCA-mediated genes that delay PD onset, prevent its progression or reverse its symptoms, and provide disease-modifying therapies, i.e., treating the actual cause of PD rather than treating mere symptoms of PD in carriers of a SNCA genomic variant.

### BRIEF DESCRIPTION OF THE DRAWINGS

FIG. 1: Non-canonical Notch pathway and involvement of PD-associated genes and SNCA-mediated genes. The functions of SNCA-mediated genes PDZRN4, ABCG2, PPM1K, HERC5 and HERC6 are placed in the non-canonical Notch biological pathway. HERC5 and HERC6 are designated as HERC5/6 in FIG. 1.

FIG. 2: DNA sequences of SNCA genomic variants linked to PD and Lewy body dementia.

FIG. 3: High scoring transcription factor motifs found in the location of SNCA genomic variants. FIG. 3A: Alanine 30 region (A30). FIG. 3B: Histidine 50-Alanine 53 region (H50-A53).

FIG. 4: Effects of rs104893878 (A30P) SNCA genomic variant on transcription factor binding motifs. FIG. 4A: Stat5A::Stat5B sequence logo and position frequency matrix. FIG. 4B: TCF3 and TCF4 sequence logo sequence. FIG. 4C: TCF3 position frequency matrix.

FIG. 5: Effects of rs201106962 (H50Q) SNCA genomic variant on transcription factor binding motifs. FIG. 5A: KLF5 sequence logo and position frequency matrix. FIG. 5B: Klf1 sequence logo and position frequency matrix (murine). FIG. 5C: Klf4 sequence logo and position weight matrix (murine).

FIG. 6: Effects of rs431905511 (G51D) SNCA genomic variant on transcription factor binding motifs. FIG. 6A: Pax2 sequence logo and position frequency matrix (murine). FIG. 6B: MAFG::NFE2L1 sequence logo and position frequency matrix (chicken).

FIG. 7: Effects of rs104893877 (A53T) SNCA genomic variant on transcription factor binding motifs. FIG. 7A: NFIX sequence logo and position frequency matrix. FIG. 7B: CEBPA sequence logo and position frequency matrix. FIG. 7C: MGA sequence logo and position frequency matrix. FIG. 7D: SREBF1 and SREBF2 sequence logo and position frequency matrix.

### DETAILED DESCRIPTION OF THE INVENTION

The common form of PD presents as late onset, and is influenced by age, medical history and genetic factors. Although seemingly “sporadic” (i.e., not following Mendelian inheritance), a large proportion of cases seems nonetheless substantially controlled by genetic factors. The familial forms of PD present as early onset and are strongly influenced by genetic factors but environmental factors may contribute to the degree of genetic penetrance of disease expression. Numerous loci with seemingly unconnected biology have been associated with PD so far (Table 1). The strongest associations with PD are for SNPs in and around the SNCA and MAPT genes. Other studies have reported association between late PD and LRRK2.

Several of the loci listed in Table 1 are linked to genetic mutations transmitted in an autosomal dominant fashion. PARK1 and PARK4 are mutations or multiplication of the alpha-synuclein gene (SNCA), PARK8 are mutations in the SNCA gene, PARK17 is a mutation in the VPS35 gene, PARK18 is a heterozygous mutation in the EIF4G1 and PARK22 is a mutation in the CHCHD2 gene. Several loci for autosomal recessive early-onset PD have been identified as well: PARK2 are mutations in the Parkin gene, PARK6 are mutations in the PINK1 gene, PARK7 are homozygous or compound heterozygous mutations in the DJ-1 gene, PARK9/Kufor-Rakeb are mutations in the ATP13A2 gene, PARK15 are mutations in the FBXO7 gene, PARK19 are mutations in the DNAJC6, PARK20 is a mutation in the SYNJ1 gene, PARK21 is a mutation in the DNAJC13 gene and PARK23 are homozygous or compound heterozygous mutations in the VPS13C gene. Mutations in PARK5/UCHL1, PARK13/HTRA2, and PARK14/PLA2G6 were reported in isolated patients.

TABLE 1
Human genetic loci associated with Parkinson's disease.

| Gene | Alias | Chromosome | Transmission | Population | Histopathology |
|---|---|---|---|---|---|
| PARK7 | DJI | lp36.23 | Autosomal recessive, early onset | 10 families | No Lewy bodies |
| ATP13A2 | PARK9 | lp36.13 | Autosomal recessive, juvenile onset | | Lewy bodies |
| | Kufor-Rakeb | | | | |
| PINK1 | PARK6 | lp36.12 | Autosomal recessive, early onset | 65 families | No Lewy bodies |
| DNAJC6 | PARK19 | lp31.3 | Autosomal recessive, juvenile onset | 2 families | No information |
| | auxilin | | | | |
| ELAV4 | PARK10 | lp34 | Susceptibility to late onset | Association studies, | No information |
| | HuD | | | multifactorial | |
| GBA | PARK16 | lq22 | Susceptibility to late onset | Association studies, | Lewy Bodies |
| | | | | multifactorial | |
| | PARK16 | lq32 | Susceptibility to late onset | Association studies | Lewy Bodies |
| HTRA2 | PARK3 | 2p13.1 | Autosomal dominant | Linkage studies | No information |
| HTRA2 | PARK13 | 2p13.1 | Association with disease not | 4 subjects | No information |
| | | | confirmed | | |
| GIGYF2 | PARK11 | 2q37.1 | Susceptibility to late onset | Association studies, | No information |
| | | | | isolated patients | |
| DNAJC13 | PARK21 | 3q22.1 | Autosomal dominant, late onset | 1 family | Lewy bodies |
| | RME-8 | | | | |
| EIF4G1 | PARK18 | 3q27.1 | Autosomal dominant, late onset, | 1 family + isolated | Lewy bodies |
| | | | low penetrance | subjects | |
| UCHL1 | PARK5 | 4p13 | Not confirmed | 2 subjects | No information |
| | | | Susceptibility to late onset | | |
| | | | | | No information |
| SNCA | PARK1 | 4q22.1 | Autosomal dominant, late onset | 50 families | Lewy bodies |
| | PARK4 | | | Association studies | Lewy bodies |
| ADH1C | | 4q23 | Susceptibility to late onset | Association studies | No information |
| PARK2 | Parkin | 6q26 | Autosomal recessive, juvenile | 607 families | No Lewy Bodies |
| CHCHD2 | PARK22 | 7p11.2 | Autosomal dominant | 1 family | No information |
| SNCA | PARK8 | 12q12 | Autosomal dominant, late onset | 940 families | Variant dependent |
| | | | Susceptibility to late onset | Association studies | Lewy bodies |
| ATXN2 | Ataxin 2 | 12q24.1 | Susceptibility to late onset | | No information |
| | SCA2 | | | | |
| VPS13C | PARK23 | 15q22.2 | Autosomal recessive, early onset | | Diffuse Lewy |
| | | | | | bodies |
| VPS35 | PARK17 | 16q11.2 | Autosomal dominant, late onset, | 3 families | No Lewy bodies |
| | | | reduced penetrance | | |
| MAPT | TAU | 17q21.31 | Susceptibility to late onset | Association studies | No information |
| TMEM230 | PARK21 | 20p13 | Autosomal dominant, late onset | Same family as | No information |
| | | | | DNAJC13 | |
| SYNJ1 | PARK20 | 21q22.11 | Autosomal recessive, early onset | 2 families | No information |
| FBXO7 | PARK15 | 22q12.3 | Autosomal recessive, early onset | 10 families | No information |
| PLA2G6 | PARK14 | 22q13.1 | Autosomal recessive, dystonia- | 2 families + isolated | No information |
| | | | parkinsonism | subjects | |
| | PARK12 | Xq21-q25 | Susceptibility to late onset | Linkage analysis | No information |

The human SNCA (alpha synuclein) gene (ENSG00000145335; chromosomal location of SNCA 4:89724099-89838315) is located on human chromosome 4 and is defined as the SNCA wild-type. SNCA has its highest expression in the brain and accounts for 1% of all proteins in the cytosol of brain cells. The normal neuronal function of SNCA is unclear but it has been suggested that it could be involved in synaptic vesicles biology. Four different SNCA genomic variants rs104893878 (A30P), rs201106962 (H50Q), rs431905511 (G51D), rs104893877 (A53T), and a SNCA gene triplication have been identified that cause PD.

SNCA genomic variants have been identified in numerous families with inheritable PD. SNCA genomic variants have also been associated with idiopathic (i.e, sporadic, late onset) Parkinson's disease^{iv}. Hence, SNCA genomic variants are linked to both idiopathic and familial PD. Examples of SNCA genomic variants linked to PD are rs104893878 (A30P), rs201106962 (H50Q), rs431905511 (G51D), rs104893877 (A53T), rs356182, rs2736990, rs2572324, rs7684318, and rs894278.

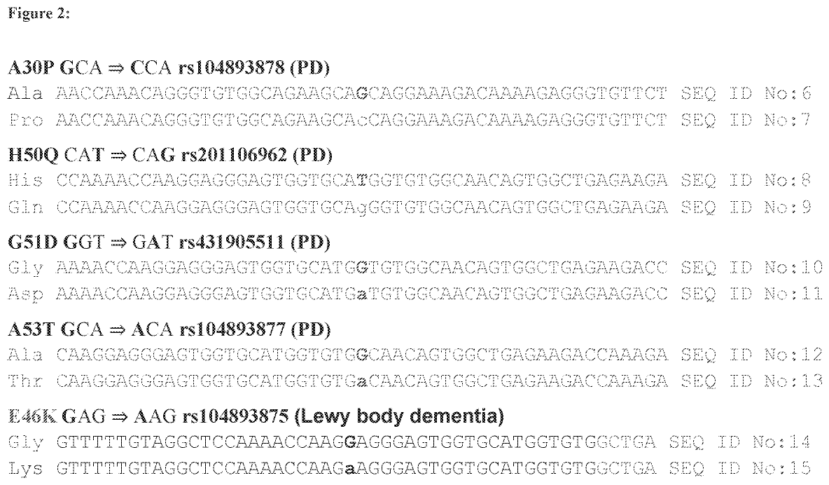

Five different pathological SNCA genomic variants have been identified that lead to neurodegeneration (FIG. 2). While rs104893878 (A30P), rs201106962 (H50Q), rs431905511 (G51D) and rs104893877 (A53T) cause Parkinson's disease, the rs104893875 (E46K) causes Lewy body dementia. SNCA gene duplication causes both PD and Lewy Body dementia, while SNCA gene triplication cause PD only. Hence overexpression of the SNCA wild-type protein and loss-of-function mutations have the same effect, a situation that is difficult to reconcile and might be indicative of a mechanism that is substantially different from an effect of the amino acid changes observed in some of the SNCA genomic variants. The rs201106962 (H50Q) SNCA genomic variant clinical picture mimics late-onset idiopathic PD. Immunoelectron microscopy of rs104893877 (A53T) tissue demonstrated fibrillar alpha-synuclein-immunoreactive aggregates and tau neuritic inclusions and less frequent perikaryal inclusions^{v}. SNCA duplication led to a clinical phenotype of frontotemporal dementia with marked behavioral changes, pyramidal signs, postural hypotension and transiently levodopa responsive parkinsonism. The genomic variant rs431905511 (G51D) shows widespread inclusions, and results in clinical and neuropathological features with similarities to multiple system atrophyi.

Penetrance of a disease-causing variant is defined as the fraction of individuals carrying this variant who manifest the disorder. Penetrance of the various SNCA variants associated with PD is also surprisingly dissimilar. The rs104893877 variant (A53T) exhibits 85% penetrance while the penetrance for SNCA duplication is reduced and can be as low as 33% in one particular family. As a comparison, homozygous or compound mutations in PARK2, PINK1 and PARK7 exhibit 100% penetrance. Reduced penetrance is difficult to reconcile with a protein-driven pathological mechanism.

Motor symptoms of PD usually appear when 50-80% of the dopaminergic neurons in the substantia nigra are lost. Triplication of the SNCA gene leads to an earlier onset, more severe phenotype than the SNCA gene duplication. Carriers of the SNCA genomic variant rs104893877(A53T) have an early age of onset (45 years), about ten years younger than carriers of other SNCA genomic variants. The mean age of onset for rs104893878 (A30P) patients is 54 years. In the three families with the rs431905511(G5TD) SNCA genomic variant, disease onset ranges from 19 to 60 years of age.

The neuropathological hallmarks of PD are the progressive loss of dopaminergic neurons in the substantia nigra pars compacta and the presence of cytoplasmic aggregates of ubiquinated, phosphorylated, truncated SNCA, called Lewy bodies. Dystrophic Lewy neurites containing alpha-synuclein filaments are observed in surviving neurons. Since genomic duplication of SNCA can cause an autosomal dominant form of PD, one simple pathological mechanism has been proposed, i.e. that overexpression (or mutation) of alpha-synuclein leads to its toxic aggregation and that this protein build-up is somehow related to cell-death. Unfortunately, a lot of observations do not support this theory. First of all, in the brain, SNCA is expressed both in regions in which Lewy Bodies deposition occurs (e.g., substantia nigra) and in less susceptible regions (e.g., visual cortex). It is also expressed in other, non-neuronal tissues (kidney, melanocytes) which show no evidence of aggregation or death. There is moreover a dissociation between the presence and density of Lewy Bodies, cellular loss and clinical disease symptoms^{vii}. Further, mutation in a given protein leads to aggregation of another one: Lewy bodies resulting from non-SNCA genomic variants also contain alpha-synuclein and ubiquitin. Thus, the presence of aggregates is not the direct consequence of mutation in a given protein but can be the consequence of mechanisms related to other genes and proteins. Finally, PINK1, PARK2, PARK7, and VPS35 post-mortem diagnoses never reveal Lewy bodies (Table 1). Thus, it is clear that nigral degeneration is not subordinate to Lewy body formation or neurofibrillary tangle formation nor any kind of aggregates.

Apart from α-synuclein aggregation, proteosomal and lysosomal dysfunction, and reduced mitochondrial activity have been proposed as cause of cell death. Although these disturbances may explain observed pathology for some of the variants, they do not offer a satisfying explanation for the disease as a whole.

Reduced penetrance, variability in impressibility both in tissues and subjects, and striking pathological differences within mutations affecting the same protein/protein domain are difficult to explain with a faulty protein function mechanism.

The present invention discloses a disease mechanism that unifies components of etiology, impairment associated with gene mutations, findings from genome wide association studies, tissue specificity of disease manifestation and environmental effects.

The present invention discloses that SNCA genomic variants cause PD through an alternative, DNA based mechanism that allows incorporation of exposure to external factors in carriers of SNCA genomic variants. Tissue specific gene expression is controlled by DNA sequences called cis-regulatory modules that can function over large genomic distances. Tissue specific gene expression is also controlled by DNA sequences acting in trans, thereby regulating gene expression of genes located on a different chromosome. Transcription factors and other DNA binding proteins bind to these regulatory elements resulting in stimulation/enhancement or alternatively repression of gene transcription. The invention discloses that SNCA genomic variants either create new enhancer or other transcription factor binding elements via de novo creation of a transcription factor binding site, or alter existing enhancers or other transcription factor binding sites that control the activity of a spatially close neighboring gene, the “SNCA-mediated gene.” The spatially close neighboring gene can be located on the same chromosome as SNCA or on a different human chromosome held in close proximity by transcription factors and/or other trans-acting proteins.

The present invention reveals that SNCA genomic variant alleles are unexpectedly modulating (e.g., activating or suppressing) transcription of a range of genes besides of SNCA itself. These genes, located in the genomic vicinity of SNCA, in both cis and trans relationships, excluding SNCA, are herein referred to as SNCA-mediated genes. These genes are not activated (or suppressed) to the same extent by the SNCA wild-type, thereby providing an explanation for the correlation of SNCA genomic variants with PD.

This DNA based mechanism allows for the incorporation of exposure to external factors in disease mutation carriers. Further, it can also accommodate the puzzling observation that SNCA genomic variants linked to PD have the same global effect as SNCA gene triplication.

Further, SNCA genomic variants create additional de novo binding sites for transcription factors that are already present in the genomic region. This is equivalent in terms of transcription factor binding sites to a gene duplication or a gene triplication. Such a disease mechanism has been uncovered in Hereditary Mixed Polyposis Syndrome (HMPS), that is caused by a 40-kb duplication. The duplicated fragment contains predicted enhancer elements that direct ectopic expression of a downstream, disease causing protein.

SNCA genomic variants are human SNCA genes that have one or more of the nucleotides present in the wild-type human SNCA gene substituted by one or more different nucleotides. In some instances, the nucleotide change results in an amino acid change in the encoded SNCA protein. In other instances, the nucleotide change does not affect the resulting amino acid sequence of the encoded SNCA protein. There are many naturally occurring human SNCA genomic variants known to the skilled in the art. For instance, rs104893878, rs201106962, rs431905511, rs104893877, rs104893875, rs356182, rs2736990, rs2572324, rs7684318, rs894278, rs76642636 and rs764216470 are examples of genomic variants in the human SNCA gene.

SNCA genomic variants linked to PD are rs104893878 (A30P), rs201106962 (H50Q), rs431905511 (G51D) and rs104893877 (A53T). These main PD causing SNCA genomic variants are apart on chromosome 4q21. Rs104893878 (A30P) is located within exon 2 and the three genomic variants rs201106962 (H50Q), rs431905511 (G51D) and rs104893877 (A53T) are on exon 3. Since exon 2 and exon 3 are very small, 146 bp and 42 bp respectively, and separated by a long intron (7362 bp) they cannot be part of the same enhancer element.

The present invention discloses a search of the DNA sequences in the vicinity of each SNCA genomic variant (FIG. 2) for the presence of transcription factor binding sites using binding profiles from the JASPAR CORE database of experimentally defined transcription factor binding sites for eukaryotes (http://jaspar.genereg.net). A relative profile score threshold cut-off of 85% was used. A score was calculated for the probed sequence that provides a measure of similarity to the transcription factor consensus sequence. A transcription factor binding site was classified as “gained” or “de novo” in a SNCA genomic variant if it is found by the JASPAR screen using the 85% threshold cut-off value and it did not occur in the screen when the SNCA wild-type sequence was used in the query. Similarly, a transcription factor binding site was classified as “lost” if it was not found using the SNCA genomic variant sequence as query with the 85% threshold cut-off value. An increase in score means that the score was higher by at least one point in the SNCA genomic variant compared to the SNCA wild-type sequence. A summary of the results is shown in Table 3. Transcription factor bonding motif position (i.e. start and end) are with respect to the DNA sequences shown in FIG. 2.

TABLE 3
Analysis of transcription factor binding sites in SNCA genomic variants.

| SNCA | JASPAR | Transcription | | | | |
|---|---|---|---|---|---|---|
| Genomic variant | Model ID | factor | Score | Start | End | Strand |
| rs104893878 | | | | | | |
| A30P | | | | | | |
| Sites gained | | | | | | |
| | MA0824.1 | ID4 | 7.306 | 22 | 31 | 1 |
| | MA0519.1 | Stat5a::Stat5b | 6.498 | 24 | 34 | −1 |
| | MA0522.2 | TCF3 | 5.798 | 22 | 31 | 1 |
| | MA0830.1 | TCF4 | 5.790 | 22 | 31 | 1 |
| Site lost | | | | | | |
| | MA0499.1 | Myod1 | | 19 | 31 | −1 |
| Increased score | | | | | | |
| | MA0136.2 | ELF5 | 9.625 | 25 | 35 | 1 |
| rs201106962 | | | | | | |
| H50Q | | | | | | |
| Site gained | | | | | | |
| | MA0742.1 | Klf12 | 11.478 | 21 | 35 | −1 |
| | MA0746.1 | SP3 | 9.026 | 24 | 34 | −1 |
| Site lost | | | | | | |
| | MA0738.1 | HIC2 | 5.442 | 18 | 26 | −1 |
| Increased score | | | | | | |
| | MA0493.1 | Klf1 | 15.659 | 25 | 35 | −1 |
| | MA0039.2 | Klf4 | 15.153 | 25 | 34 | 1 |
| | MA0599.1 | KLF5 | 13.531 | 25 | 34 | −1 |
| | MA0801.1 | MGA | 7.320 | 26 | 33 | 1 |
| | MA0806.1 | TBX4 | 7.095 | 26 | 33 | 1 |
| | MA0807.1 | TBX5 | 8.183 | 26 | 33 | 1 |
| rs431905511 | | | | | | |
| G51D | | | | | | |
| Site gained | | | | | | |
| | MA0067.1 | Pax2 | 6.639 | 21 | 28 | −1 |
| | MA0089.1 | MAFG::NFE2L1 | 6.932 | 22 | 27 | 1 |
| Sites lost | | | | | | |
| | MA0493.1 | Klf1 | | 23 | 33 | −1 |
| | MA0039.2 | Klf4 | | 23 | 32 | 1 |
| | MA0599.1 | KLF5 | | 23 | 32 | −1 |
| | MA0801.1 | MGA | | 24 | 31 | 1 |
| | MA0806.1 | TBX4 | | 24 | 31 | 1 |
| | MA0807.1 | TBX5 | | 24 | 31 | 1 |
| rs104893877 | | | | | | |
| A53T | | | | | | |
| Sites gained | | | | | | |
| | MA0800.1 | EOMES | 11.647 | 18 | 30 | 1 |
| | MA0032.2 | FOXC1 | 6.931 | 22 | 32 | 1 |
| | MA0847.1 | FOXD2 | 6.127 | 25 | 31 | 1 |
| | MA0849.1 | FOXO6 | 5.315 | 25 | 31 | 1 |
| | MA0067.1 | Pax2 | 6.345 | 21 | 28 | −1 |
| | MA0774.1 | MEIS2 | 7.258 | 23 | 30 | 1 |
| | MA0775.1 | MEIS3 | 8.712 | 23 | 30 | 1 |
| | MA0595.1 | SREBF1 | 11.951 | 18 | 27 | −1 |
| | MA0596.1 | SREBF2 | 13.052 | 18 | 27 | 1 |
| | MA0802.1 | TBR1 | 9.166 | 19 | 28 | 1 |
| | MA0805.1 | TBX1 | 10.681 | 19 | 26 | 1 |
| | MA0803.1 | TBX15 | 9.024 | 19 | 26 | 1 |
| | MA0688.1 | TBX2 | 10.612 | 18 | 28 | 1 |
| | MA0690.1 | TBX21 | 9.291 | 18 | 27 | 1 |
| | MA0088.2 | ZNF143 | 9.957 | 12 | 27 | −1 |
| Site lost | | | | | | |
| | MA0102.3 | CEBPA | 7.992 | 20 | 30 | −1 |
| | MA0493.1 | Klf1 | 9.173 | 18 | 28 | −1 |
| | MA0039.2 | Klf4 | 7.588 | 18 | 27 | 1 |
| | MA0599.1 | KLF5 | 4.713 | 18 | 27 | −1 |
| | MA0671.1 | NFIX | 6.937 | 22 | 30 | 1 |
| | MA0671.1 | NFIX | 5.382 | 22 | 30 | −1 |
| Increased score | | | | | | |
| | MA0806.1 | TBX4 | 9.638 | 19 | 26 | 1 |
| | MA0807.1 | TBX5 | 9.595 | 19 | 26 | 1 |
| | MA0801.1 | MGA | 11.216 | 19 | 26 | 1 |
| | MA0498.2 | MEIS1 | 8.174 | 23 | 29 | 1 |
| | MA0848.1 | FOXO4 | 6.700 | 25 | 31 | 1 |

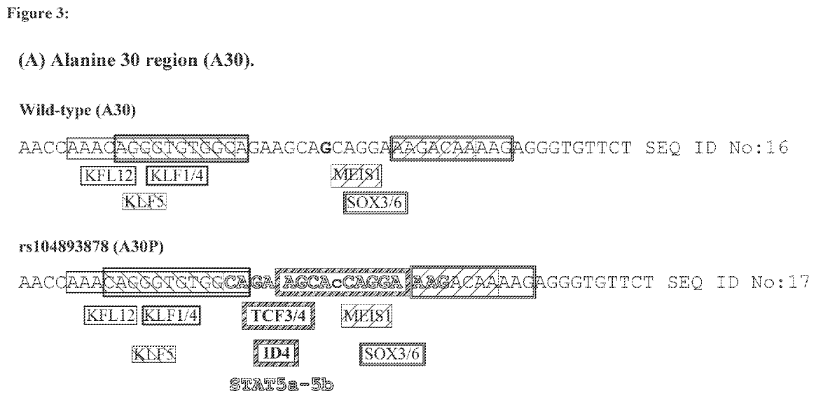

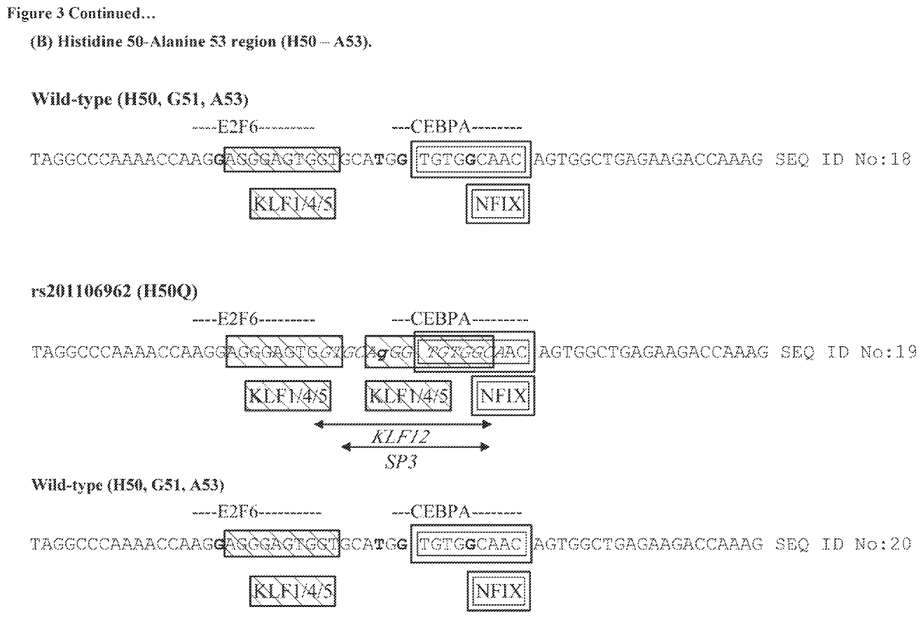

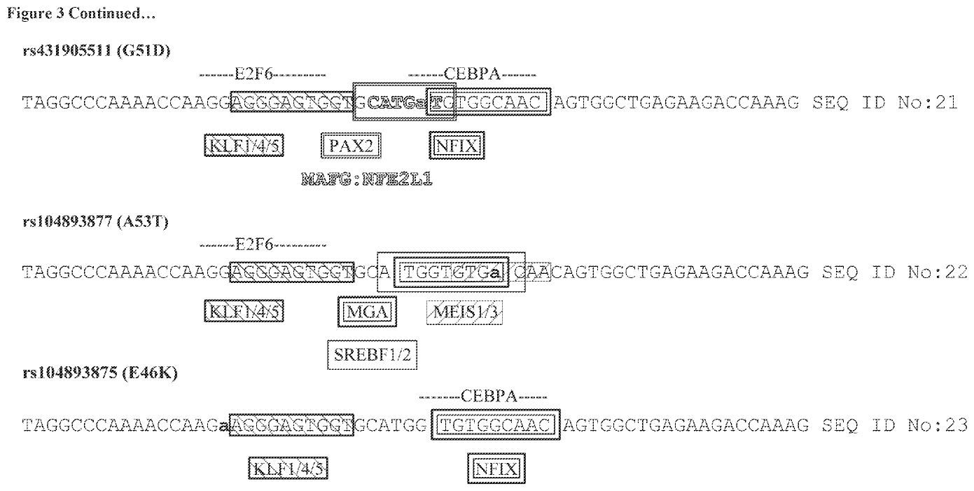

FIG. 3 shows the highest scoring transcription factor binding sites found in 50 bp stretches overlapping SNCA genomic variants. The rs104893878 variant generates a motif for TCF3, ID4 and Stat5a::Stat5b. The rs201106962 variant creates an additional motif for KLF1/4/5 next to one already present. Further, it creates a de novo motif for KLF12. The rs431905511 variant creates a motif for PAX2 and MAFG::NFE2L1. The rs104893877 variant creates motifs for MGA and MEISI1/3 while removing motifs for CEBPA and NFIX. The transcription factor CEBPA intersects with the Notch signaling pathway. CCAAT/enhancer-binding protein (CEBP) transcription factors are critical for gene expression in tissue development and cellular function, proliferation, and cell differentiation.

The present invention discloses that SNCA genomic variants alter transcription factor recognition motifs and act as enhancers causing improper transcription of one or several of SNCA-mediated genes in PD vulnerable neurons in carriers of SNCA genomic variants.

The most common means of transcriptional regulation occurs in cis, as 98% of chromatin loops anchored at a promoter are located within a range of 2 Mb of the enhancer's location, indicating that the vast majority of genes regulated by the enhancer are located within 2 Mb of the enhancer's chromosomal position^{viii}. Studies from a variety of organisms suggest that mutations affecting cis-regulatory activity are the predominant source of expression divergence between species. In some embodiments, “SNCA-mediated gene” means any gene located within 2 Mb (2 megabases=2 million bases) upstream or downstream of the location of SNCA (ENSG00000145335; chromosomal location of SNCA 4:89724099-89838315).

Transcriptional regulation emanating from enhancer elements can also function in trans, meaning that they can regulate the expression of genes in chromosomes distinct from the chromosome of the transcriptional enhancer element. In a further embodiment, SNCA-mediated genes are located on human chromosome 12 and include PDZRN4.

Preferably, the SNCA-mediated gene is selected from the group consisting of KLHL8, PTPN13, HSD17B13, HSD17B11, NUDT9, SPARCL1, DMP1, IBSP, MEPE, SPP1, PKD2, ABCG2, PPM1K, HERC6, HERC5, PIGY, PYURF, HERC3, NAP1L5, FAM13A, TIGD2, GPRIN3, SNCA-AS1, MMRN1, CCSER1, GRID2, PDZRN4, or a combination thereof. More preferably, the SNCA-mediated gene is selected from the group consisting of ABCG2, PPM1K, HERC3, HERC5, HERC6, SPARCL1, MMRN1, PDZRN4, or a combination thereof. Most preferably, the SNCA-mediated gene is selected from the group consisting of HERC5, HERC6, SPARCL1, MMRN1, PDZRN4, or a combination thereof.

Still in other embodiments, the term “SNCA-mediated gene” or the “expression product” refers to the gene or the expression product, respectively, that is modulated (e.g., either increased or decreased) by the presence of SNCA genomic variants. In some cases, the activity of the SNCA-mediated gene or the expression product thereof is increased in SNCA genomic variants compared to SNCA wild-type. In other cases, the activity of the gene or the expression product thereof is reduced in SNCA genomic variants compared to SNCA wild-type. A gene expression product comprises a transcription product of such a gene including any RNA transcript based on such gene, including any microRNA or mRNA (whether the mRNA transcript is primary, spliced, edited, modified or mature) or a polypeptide translated from an mRNA transcript of an SNCA-mediated gene. Such polypeptide may be nascent or processed into a mature or modified form of the protein.

A representative list of the SNCA-mediated genes located on chromosome 4 in about a 2 Mb window upstream or downstream of the location of SNCA (ENSG00000145335; chromosomal location of SNCA 4:89724099-89838315), was compiled from the GRCh38.p5 Homo sapiens Genome Assembly of the Ensembl genome database and is provided in Table 4.

TABLE 4
Representative list of genes located within about a 2 Mb window
upstream or downstream of the location of SNCA (ENSG00000145335;
chromosomal location of SNCA 4:89724099-89838315), was compiled
from the GRCh38.p5 Homo sapiens Genome Assembly
of the Ensembl genome database. SNCA-AS1 is a gene distinguished
from SNCA that is transcribed from the opposite strand of the genome.

| Gene Symbol | Gene ID | Chromosomal location |
|---|---|---|
| KLHL8 | ENSG00000145332 | 4:87160103-87240314 |
| PTPN13 | ENSG00000163629 | 4:86594315-86815171 |
| HSD17B13 | ENSG00000170509 | 4:87303789-87322906 |
| HSD17B11 | ENSG00000198189 | 4:87336610-87391386 |
| NUDT9 | ENSG00000170502 | 4:87422582-87459454 |
| SPARCL1 | ENSG00000152583 | 4:87473335-87531061 |
| DMP1 | ENSG00000152592 | 4:87650307-87664361 |
| IBSP | ENSG00000029559 | 4:87799581-87812435 |
| MEPE | ENSG00000152595 | 4:87821411-87846817 |
| SPP1 | ENSG00000118785 | 4:87975650-87983426 |
| PKD2 | ENSG00000118762 | 4:88007668-88077777 |
| ABCG2 | ENSG00000118777 | 4:88090264-88231322 |
| PPM1K | ENSG00000163644 | 4:88257620-88284769 |
| HERC6 | ENSG00000138642 | 4:88378739-88443111 |
| HERC5 | ENSG00000138646 | 4:88457117-88506163 |
| PIGY | ENSG00000255072 | 4:88521573-88521789 |
| PYURF | ENSG00000145337 | 4:88520985-88523813 |
| HERC3 | ENSG00000138641 | 4:88521048-88708542 |
| NAP1L5 | ENSG00000177432 | 4:88695915-88698235 |
| FAM13A | ENSG00000138640 | 4:88725955-89111398 |
| TIGD2 | ENSG00000180346 | 4:89111500-89114899 |
| GPRIN3 | ENSG00000185477 | 4:89236386-89308010 |
| SNCA-AS1 | ENSG00000247775 | 4:89836408-89841978 |
| MMRN1 | ENSG00000138722 | 4:89879532-89954629 |
| CCSER1 | ENSG00000184305 | 4:90127535-91601913 |
| GRID2 | ENSG00000152208 | 4:92303622-93774556 |

Transcription factor binding motifs affected by the SNCA genomic variants are either present in proximal enhancers or the promoter regulating the SNCA-mediated genes lying in the vicinity of the SNCA genomic variants (Table 5). These motifs may represent weak affinity binding sites (the score obtained by comparing the position weight matrix of a transcription factor binding motif against the target sequence is proportional to the binding energy between this transcription factor and the target DNA). These sites can influence gene regulation as non-consensus, weak transcription factor binding. In certain gene enhancers this is specifically required for proper responses. This is especially true for Hedgehog or Notch signaling.

TABLE 5
Correlation between transcription factors binding motifs, regulation
of candidate genes and Parkinson's disease pathology.

| SNCA | | | | |
|---|---|---|---|---|
| Genomic | | | | |
| variant | Transcription factor | Candidate gene | Candidate gene effect | Outcome |
| rs104893878 | ID4 | HERC6 | PARK2 | Mitochondrial dysfunction |
| A30P | ID4 (trans) | PDZRN4 | Notch regulation | Lewy Bodies (autophagy) |
| | TCF3/4 | HERC5 HERC6 | PARK2 | Mitochondrial dysfunction |
| | TCF3/4 (trans) | HERC3 | PARK2, UBQLN1 | Lewy Bodies (autophagy) |
| | | SPARCL1 | Dendrites formation | Dendritic arbor dystrophy |
| | | PPM1K | PINK1 | Mitochondrial permeability |
| | | PDZRN4 | Notch regulation | Mitochondrial dysfunction |
| | Stat5a::Stat5b | PPM1K | PINK1 | Mitochondrial permeability |
| | | HERC5 HERC6 | PARK2 | Mitochondrial dysfunction |
| | | HERC3 | PARK2, UBQLN1 | Lewy bodies (autophagy) |
| rs201106962 | SP3 | HERC5 | PARK2 | Mitochondrial dysfunction |
| H50Q | | | | Lewy Bodies (autophagy) |
| | KLF4, KLF5 (trans) | ATOH1 | Notch pathway | Olfactory and goblets dysfunction |
| | KLF4, KLF5 | HERC5 | PARK2 | Mitochondrial dysfunction |
| | | PPM1K | PINK1 | Lewy Bodies (autophagy) |
| | | | | Mitochondrial permeability |
| | KLF5 | MMRN1 | Synaptic scaling | Synaptic homeostasis |
| | KLF1 | PPM1K | PINK1 | Mitochondrial permeability |
| | | ABCG2 | Mitochondrial fission | Mitochondrial dysfunction |
| | TBX4/5 | HERC5 HERC6 | PARK2 | Mitochondrial dysfunction |
| | TBX4/5 (trans) | PDZRN4 | Notch regulation | Lewy Bodies (autophagy) |
| | | | | Mitochondrial dysfunction |
| rs431905511 | Pax2 | HERC3 HERC5 | PARK2, UBQLN1 | Mitochondrial dysfunction |
| G51D | | SPARCL1 | Dendrites formation | Lewy bodies (autophagy) |
| | | MMRN1 | Synaptic scaling | Dendritic arbor dystrophy |
| | | | | Synaptic homeostasis |
| | MAFG::NE2FL1 | HERC3 | PARK2 | Mitochondrial dysfunction/LB |
| | | ABCG2 | Mitochondrial fission | Mitochondrial dysfunction |
| | | PPM1K | PINK1 | Mitochondrial permeability |
| | | MMRN1 | Synaptic scaling | Synaptic homeostasis |
| rs104893877 | EOMES | ABCG2 | Mitochondrial fission | Mitochondrial dysfunction |
| A53T | EOMES (trans) | PDZRN4 | PINK1 | Mitochondrial permeability |
| | FOXD2 | HERC3 HERC6 | PARK2 | Mitochondrial dysfunction |
| | FOXO4/FOXO6 | ABCG2 | Mitochondrial fission | Lewy bodies (autophagy) |
| | | PPM1K | PINK1 | Mitochondrial dysfunction |
| | | SPARCL1 | Dendrites formation | Mitochondrial permeability |
| | | MMRN1 | Synaptic scaling | Dendritic arbor dystrophy |
| | | | | Synaptic homeostasis |
| | Pax2 | HERC3 HERC5 | PARK2, UBQLN1 | Mitochondrial dysfunction |
| | | SPARCL1 | Dendrites formation | Lewy bodies (autophagy) |
| | | MMRN1 | Synaptic scaling | Dendritic arbor dystrophy |
| | | | | Synaptic homeostasis |
| | SREBF2 | HERC6 | PARK2 | Mitochondrial dysfunction |
| | | PPM1K | PINK1 | Lewy Bodies (autophagy) |
| | MGA | HERC6 | PARK2 | Mitochondria dysfunction |
| | MGA (trans) | PDZRN4 | Notch regulation | Lewy Bodies (autophagy) |
| | | | | Mitochondrial dysfunction |
| | TBX4/5 | HERC5 HERC6 | PARK2 | Mitochondrial dysfunction |
| | TBX4/5, TBX1, TBX15 | PDZRN4 | Notch regulation | Lewy Bodies (autophagy) |
| | (trans) | | | |
| | MEIS2 | ABCG2 | Mitochondrial fission | Mitochondrial dysfunction |
| | | PPM1K | PINK1 | Lewy Bodies (autophagy) |
| | | MMRN1 | Synaptic scaling | Synaptic homeostasis |
| | MEIS1/3 | HERC3 | PARK2, UBQLN1 | Mitochondrial dysfunction |
| | | HERC5 HERC6 | PARK2 | Lewy bodies (autophagy) |
| | | ABCG2 | Mitochondrial fission | Mitochondrial dysfunction |
| | | PPM1K | PINK1 | Lewy bodies (autophagy) |
| | | SPARCL1 | Dendrites formation | Mitochondrial dysfunction |
| | | | | Mitochondrial permeability |
| | | | | Dendritic arbor dystrophy |
| | ZNF143 | HERC5 HERC6 | PARK2 | Mitochondrial dysfunction |
| | ZNF143 (trans) | HERC3 | PARK2, UBQLN1 | Lewy bodies (autophagy) |
| | | ABCG2 | Mitochondrial fission | Mitochondrial dysfunction |
| | | PPM1K | PINK1 | |
| | | PDZRN4 | Notch regulation | |

SNCA genomic variants can be directly anticipated to regulate SNCA-mediated genes (Table 5). To this end we performed a thorough examination of several public databases of transcription factor target genes and conducted a JASPAR analyses of 5′-sequences for each of the genes of interest as functional transcription factor binding sites are likely to be observed between 100 bp downstream and about 200 bp upstream from the transcription start site but can sometimes be found further away.

For instance, the rs104893878 variant (A30P) by its effects on transcription factors ID4, TCF3/4 and Stat5a::Stat5b regulates HERC5, HERC6 and PPM1K and SPARCL1 while its effects on transcription factors ID4 and TCF4 regulate PDZRN4 in trans.

Variant rs201106962 (H50Q) by its effects on transcription factors KLF4, KLF5 and KLF1 regulates ATOH1, HERC5, ABCG2, PPM1K and MMRN1. It also regulates HERC5 and HERC6 by its effect on SP3 and transcription factors TBX4 and 5. Moreover its effects on transcription factors MGA and TBX4/5 regulate PDZRN4 in trans.

The variant rs431905511 (G51D) through PAX2 regulates HERC3 and HERC5, SPARCL1 and MMRN1 and through MAFG/NE2FL1 regulates HERC3, ABCG2, PPM1K and MMRN1.

Variant rs104893877 (A53T) regulates ABCG2 in cis and PDZRN4 in trans through the creation of a recognition motif for EOMES. HERC3, HERC5 and HERC6, ABCG2, PPM1K, MMRN1 and SPARCL1 are regulated through the creation of de novo binding sites, e.g. FOXD2, FOXO6, Pax2, MEIS2/3 and SREBF2.

Variant rs104893877 (A53T) also increases binding site affinity for MAG, TBX4/5, MEIS1 which also regulate HERC3, HERC5, HERC6, SPARCL1, ABCG2 and PPMK1 and MMRN1 in cis and PDZRN4 in trans. PDZRN4 is also regulated by the creation of new binding motifs for TBX1, TBX15 and ZNF143.

The de novo binding motifs created by the SNCA variants sometimes overlap already existing binding motifs. As an example, Pax2 overlaps an already existing NFIX binding site (FIG. 3) which can impede NFIX binding due to competition for the same DNA region. NFIX regulates the expression of SPARCL1, PPM1K, HERC3, HERC5, HERC6, ABCG2, PPM1K, and PDZRN4 in trans. The present invention discloses that SNCA genomic variants affecting binding sites for transcription factors control the expression of SNCA-mediated genes.

Mammalians, including humans, possess four different Notch receptors, referred to as NOTCH1 (Notch Receptor 1; UniProtKB labelled as “Neurogenic locus notch homolog protein 1”, “NOTC1_HUMAN” with the accession number P46531), NOTCH2 (Notch Receptor 2; UniProtKB labelled as “Neurogenic locus notch homolog protein 2”, “NOTC2_HUMAN” with the accession number Q04721), NOTCH3 (Notch Receptor 3; UniProtKB labelled as “Neurogenic locus notch homolog protein 3”, “NOTC3_HUMAN” with the accession number Q9UM47) and NOTCH4 (Notch Receptor 4; UniProtKB labelled as “Neurogenic locus notch homolog protein 4”, “NOTC4_HUMAN” with the accession number Q99466). The term “Notch” refers herein to any of the four different Notch receptors. The Notch pathway is an evolutionarily conserved signaling pathway that mediates short-range cell-to-cell communication through interaction of membrane-tethered ligands presented by the signal-sending cell and the Notch receptor inserted in the membrane of the signal receiving cell. Notch is essential for development of many cell types including the nervous system, immunity and adult tissue homeostasis. Recently Notch has also been reported to be active in the adult brain where it influences long-term synaptic plasticity and memory^{ix}. Every step of the Notch signaling pathway is highly regulated. Its dysfunction is associated with several hereditary human diseases such as cerebral autosomal dominant arteriopathy with subcortical infarcts and leukoencephalopathy (CADASIL), Alagille syndrome, spondylocostal dysostosis and many cancers.

Canonical Notch signaling relies on a proteolytic cascade involving gamma-secretase to release Notch transcriptionally active intracellular domain (NICD) which travels into the nucleus where it forms a transcription complex with other proteins (e.g., RBJD). This transcriptional complex regulates the transcription of downstream canonical target genes, such as HES1 and HEY1.

Notch signaling can also occur in the absence of transcriptional activation through protein-protein interactions. In this pathway the Notch intracellular domain does not translocate into the nucleus. Instead, Notch acts in a non-transcriptional fashion through interaction with kinases. This pathway can be either dependent or independent of ligand binding and/or gamma-secretase processing. These pathways referred to as ‘non-canonical’ Notch signaling^{x}.

This non-canonical Notch pathway is mostly involved in nervous system gene expression and development. A ligand-dependent, but γ-secretase-independent, non-canonical Notch signaling has been evidenced in the expression of synaptic vesicle proteins in excitatory neurons.

Notch1 can be detected at the outer membrane of the mitochondria thus linking the non-canonical Notch signaling to the mitochondrial surface^{xii}. Importantly, a Notch-activated signaling cascade has been evidenced that interacts with mitochondrial remodeling proteins to regulate cell survival. Ligand processing was shown necessary, albeit canonical interactions with nuclear factors were not. In this pathway, Notch activates mechanistic target of rapamycin complex 2 (mTORC2)/Akt signaling. Notch-Akt-mediated antiapoptotic activity requires Mitofusin 1 & 2.

Akt refers to three closely related serine/threonine-protein kinases with overlapping roles, namely AKT1 (AKT Serine/Threonine Kinase 1; UniProtKB labelled as RAC-alpha serine/threonine-protein kinase, “AKT1_HUMAN” with the accession number P31749), AKT2 (AKT Serine/Threonine Kinase 2; UniProtKB labelled as RAC-alpha serine/threonine-protein kinase “AKT2_HUMAN” with the accession number P31751) and AKT3 (AKT Serine/Threonine Kinase 3; UniProtKB labelled as RAC-alpha serine/threonine-protein kinase “AKT3_HUMAN” with the accession number Q9Y243). The term “Akt” refers herein to any of the three different Akt kinases.

Several of the non-motor features of PD such as an increased number of Goblet cells in intestine and nasal epithelium and olfactory dysfunction can be directly linked to Notch inhibition. Further, Akt linked mitochondrial dysfunction as well as the observed diminished levels of both total and active phospho-Ser⁴⁷³-Akt, key molecular events in PD, can directly be correlated with the Notch pathway^{xii}. The present invention discloses an examination of the function of the genes in which familial PD mutations occur, and a review of the effects of each mutation. The invention shows that they all take part in this pathway (as recapitulated in Table 2, and exposed in the paragraphs below). For some of these PD-associated genes, involvement in the pathway has been demonstrated in drosophila (e.g., DNAJC6 or DNAJC13). Others are direct Notch interactors (PINK1) or interactors of key members of the pathway (PARK2).

TABLE 2
Genes associated with Parkinson's disease
are involved in the Notch pathway.

| Gene | Notch pathway involvement |
|---|---|
| PARK7 | Mitochondrial function/Akt phosphorylation |
| ATP13A2 | Interacts with AAK1 |
| | AAK1 phosphorylates NUMB, regulates CME |
| PINK1 | Interacts with Notch to influence mitochondrial function |
| | Activate mTORC2/AKT signaling |
| DNAJC6 | Necessary for Notch ligand Delta endocytosis and |
| | signaling in Drosophila |
| ELAV4 | Notch mRNA transport |
| PARK3 | Notch pathway members in region: AAK1, EXOC6B, |
| | RAB11FIP5 |
| DNAJC13 | Notch receptor endocytosis/recycling |
| EIF4G1 | Notch 3′ RNA processing negatively regulated to control |
| | Notch signaling in Drosophila |
| ADH1C | Role at the DNA level (gene transcription) |
| | EIF4E situated 0.26 Mb upstream is a target of Notch. |
| PARK2 | Part of a SCF-like complex, consisting of PARK2, CUL1 |
| | and FBXW7. |
| | FBXW7-dependent degradation of Notch |
| CHCHD2 | Interacts with Notch transcription regulator RBPJ |
| SNCA | Role at the DNA level (gene transcription) |
| | This present invention |
| ATXN2 | ATXN2 binds ATXN1 and ATXN1 inhibits Notch activity |
| | in mammalian cells (CBF1 corepressor) |
| VPS13C | Mitochondrial integrity |
| VPS35 | Mitochondrial trafficking |
| | Retromer-dependent recycling of Jag1 |
| MAPT | Role at the DNA level (gene transcription) |
| | CNTNAP1 in vicinity |
| SYNJ1 | Clathrin mediated Ligand endocytosis |
| | Interacts with auxilin and causes a similar early-onset |
| | phenotype |
| FBXO7 | Mitochondrial maintenance through interaction |
| | with PINK1 and Parkin |

In the adult brain, Notch signaling influences axon guidance, morphogenesis of dendritic arbors, long-term synaptic plasticity, memory and survival, mainly through its non-canonical pathway. In postmitotic neurons, Notch1 appears to be enriched in the dendrites and cell soma, while ligand Jagged1 is enriched in axons. Moreover, Notch is likely to be stored in RNA-granules in the dendrite as activity-induced Notch1 expression occurred in the presence of the transcriptional inhibitor actinomycin-D^{I}.

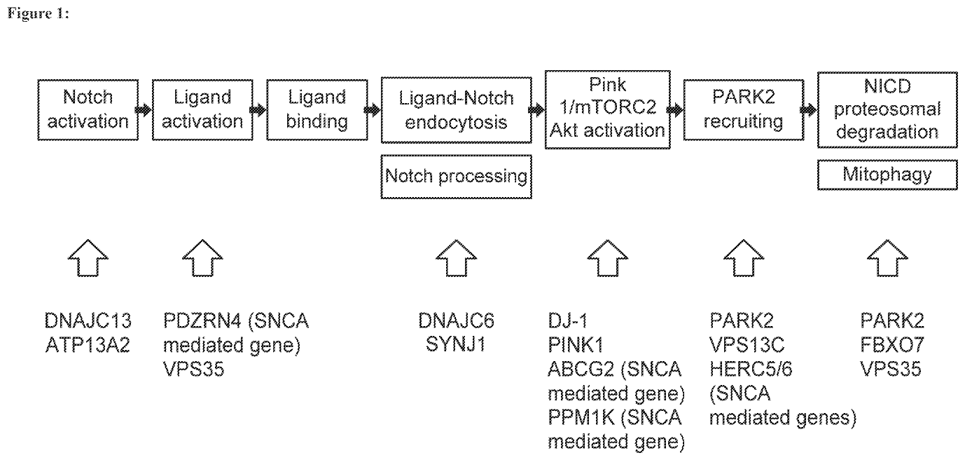

Under the induction of synaptic activity (or other triggers) in healthy neurons of the substantia nigra, Notch mRNA that had been stored as RNA granules in the dendrite of the signal receiving cell is translated, processed and activated by glycosylation and fucosylation. The resulting transmembrane receptor is tethered in the dendrite membrane, where it interacts with its receptor, expressed on the axon of the signal giving cell. Ligand binding followed by endocytosis in the signal-giving cell allows proteolytic processing of the extracellular domain by TACE, followed by release of the Notch intracellular domain (NICD) by gamma-secretase. Notch is then recruited to the mitochondria, where it binds PINK1, which in turn activates mTORC2/Akt to preserve mitochondrial integrity and protect the cell against apoptosis. To circumvent sustained activation, NICD turnover is critical. Hence phosphorylation by CDK8 promotes recognition of and proteosomal degradation of NICD by the E3 ligase FBXW7. Concomitantly phospho-Ser293-Akt leads to the phosphorylation of Hexokinase-1 which permits recruiting of Parkin/FBXO7 to the depolarized mitochondria, ubiquitination of mitofusins, and retromer assisted Akt degradation and mitophagy. The present invention discloses that genes associated with PD play roles in normal Notch function and interfere with this pathway in PD neurons of the substantia nigra when mutated. Thus, Parkinson's disease can be due to a perturbation of the non-canonical Notch pathway (FIG. 1).

The present invention discloses that several SNCA-mediated genes can be placed in the non-canonical Notch pathway through their functions in endocytic trafficking, ligand binding and mitochondrial action. These genes include ABCG2, PPM1K, HERC3, HERC5, HERC6 and PDZRN4. Moreover, PD-associated mutations and some of the SNCA-mediated genes affecting intracellular routing of the Notch receptor are systematically associated with Lewy bodies.

In the adult brain, Notch signaling influences axon guidance, morphogenesis of dendritic arbors, long-term synaptic plasticity, memory and survival, mainly through its non-canonical pathway. HERC3, HERC5 and HERC6 are E3 ubiquitin-protein ligases. HERC3 is a binding partner of ubiquilin 1 and 2. Ubiquilin 1 binds and regulates autophagosome formation. Improper regulation of HERC3 will affect lysosomes/autophagosomes formation and disturb Notch recycling and activation, ultimately impeding Notch/Pink1/Mtor activation. HERC5 and HERC6 function as an interferon-induced E3 protein ligase that mediate ISGylation and activation of PARK2. Dysregulation of these genes will cause improper PARK2 activation and will lead to the premature degradation of mitochondria, before AKT activation can take place. SPARCL1 is a matricellular protein that plays a role in dendritic arborization and excitatory synaptogenesis. SPARCL1 can activate the WNT/β-catenin signaling, a pathway that intersects with Notch (presenilins). Dysregulation of ABCG2 and PPM1K expression will cause mitochondrial damage, impeding Notch/Pink1/Mtor activation.

The present invention is based on the discovery of detailed knowledge of the underlying genetic mechanism for development of PD as discussed above. In particular, such a discovery by the present invention has provided methods for more accurate diagnosis and/or treatment for PD in carriers of a SNCA genomic variants. This discovery also provides methods for preventing development of Parkinson's disease (PD) in individuals, particular those individuals that are carriers of a SNCA genomic variant.

Based on the analysis of the gene structure, the present invention discloses that SNCA genomic variants create de novo recognition motifs for transcription factors. Based at least in part by this discovery, one aspect of the invention provides a method of modulating the function of these transcription factor recognition sites created by the SNCA genomic variants to inter alia stabilize or improve the motor function and other disease manifestations of subjects with PD who are carriers of a SNCA genomic variant.

In some embodiments, modulation of SNCA-mediated gene expression is achieved by contacting a cell that is expressing a gene mediated by a SNCA genomic variant with a molecule that is capable of modulating binding of a transcription factor to the SNCA genomic variant. In some embodiments, the cell is a neuronal cell, a neuronal progenitor cell, a differentiated neuron, an oligodendrocyte, a fibroblast, a human embryonic kidney cell or another cell commonly used in drug discovery and development efforts known to one skilled in the art.

Still in another embodiment the invention provides a method for modulating the activity of a SNCA-mediated gene. It should be appreciated that the term “modulating” when referring to measuring the activity of a SNCA-mediated gene means the level of activity is modulated (i.e., increased or decreased) by at least about 10%, typically by at least about 20%, or substantially completely (i.e., >90% reduction or 10-fold increase) in the presence of a molecule of the invention compared to the same cell line in the absence of the molecule. One skilled in the art can readily measure the degree of modulation using any of the methods that are known.

The molecule used for modulating the expression or the activity of a SNCA-mediated gene includes a molecule known to one skilled in the art, such as, but not limited to, small molecules, oligonucleotides (including short interfering RNAs, RNAs, long non-coding RNAs and aptamers), peptides, polypeptides (including aptamers, zinc fingers and fragments thereof), proteins (including antibodies and fragments thereof) as well as derivatives or modified forms thereof. In some embodiments, a molecule is delivered using a viral vector. In one instance, the oligonucleotide is a single-strand oligonucleotide. Yet in another instance, the oligonucleotide is a double-strand oligonucleotide.

In one embodiment, the molecule is a small molecule. A small molecule is an organic compound with a molecular weight of less than 2000 Dalton, preferably less than 1500 Dalton and most preferably 900 Dalton or less.

In another embodiment, the molecule is an oligonucleotide. The term “oligonucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally-occurring nucleotides. Examples of such analogs include, without limitation, phosphorothioates, phosphoramidates, methyl phosphonates, chiral-methyl phosphonates, 2-O-methyl ribonucleotides, a locked-nucleic acid-modified oligonucleotide, and peptide-nucleic acids (PNAs). As used herein, the term “locked nucleic acid” refers to a nucleic acid in which the ribose moiety is modified with an extra bridge connecting the 2′-oxygen and 4′-carbon. A “subsequence” or “segment” refers to a sequence of nucleotides that comprise a part of a longer sequence of nucleotides.

In another embodiment, the oligonucleotide is fused to a cell penetrating peptide or is a peptide nucleic acid oligonucleotide, a locked-nucleic acid-modified oligonucleotide, and combinations thereof.

In one embodiment, an oligonucleotide is a ribonucleotide polymer also known as ribonucleic acid (RNA). In some instances, the RNA is a short interfering RNA (siRNA), a messenger RNA (mRNA), a microRNA (miRNA) or a long non-coding RNA (lncRNA).

In a further embodiment, the molecule is a peptide, a polypeptide or a protein. A peptide is a compound in which amino acids, including natural, artificial and modified amino acids, are linked by a peptide bond. In some embodiments, the peptide contains two to twenty linked amino acids. In another embodiment the peptide is called a polypeptide and contains up to fifty linked amino acids. In a further embodiment, the peptide contains more than fifty linked amino acids and is called a protein. In some instances, the protein is a SNCA-mediated gene expression product or a derivative thereof. In some instances, the protein is an antibody or a fragment thereof. In some instances, the peptide, the polypeptide or the protein binds to the SNCA genomic variant, more preferably to a sequence of SEQ ID NO:1, SEQ ID NO:2, SEQ ID NO:3, SEQ ID NO:4 or SEQ ID NO:5 or a combination thereof, most preferably to a segment of 9 to 30 nucleotides within the of SEQ ID NO:1, SEQ ID NO:2, SEQ ID NO:3, SEQ ID NO:4 or SEQ ID NO:5 or a combination thereof. In some instances, the peptide, the polypeptide or the protein is a zinc finger protein or a fragment thereof. In some instances, the protein is an antibody that binds to a SNCA-mediated gene expression product or a derivative thereof. In some instances, said protein that is an antibody or a fragment thereof, binds to a SNCA-mediated gene expression product selected from MMRN1, SPARCL1, or a combination thereof.

The molecule can be provided in a pharmaceutically acceptable carrier. The terms “pharmaceutically acceptable carrier” and “pharmaceutically acceptable excipient” are used interchangeably herein and refer to an excipient that is useful in preparing a pharmaceutical composition that is generally safe, non-toxic and neither biologically nor otherwise undesirable, and includes excipient that is acceptable for human pharmaceutical use.

In some embodiments, the molecule is delivered by a viral vector. In some embodiments, the viral vector is based on an adeno-associated virus, a retrovirus, a lentivirus, an adenovirus or a herpes simplex virus. In some embodiments, the viral vector encodes a protein including but not limited to an expression product of a SNCA-mediated gene or a fragment thereof, an antibody or a fragment thereof, or a zinc finger protein or a fragment thereof. In another embodiment, the viral vector encodes an RNA, including but not limited to a siRNA, a mRNA, a miRNA or a lncRNA.

Still in other embodiments, the cell comprises a neuronal cell, a neuronal progenitor cell, a differentiated neuron, an oligodendrocyte, a fibroblast, a human embryonic kidney cell or another cell commonly used in cell based assays in drug discovery and development efforts known to one skilled in the art. Methods of the cell based assays of the invention are applicable to a neuronal cell, a neuronal progenitor cell, a differentiated neuron, an oligodendrocyte, a fibroblast, a human embryonic kidney cell or another cell commonly used in drug discovery and development efforts known to one skilled in the art.

The invention provides methods to identify a lead candidate for drug development for treatment of PD in carriers of a SNCA genomic variant. The method can involve an in vitro or a biochemical assay that does not contain whole cells. The assay may contain cell extracts or other cellular components. The assay may also comprise a substantially purified gene or gene expression product. In this context “substantially purified” refers to a composition where the gene or gene expression product is present at least 10-fold higher than in any naturally occurring context, and wherein elements of the natural context have been removed, yet wherein the gene or gene expression product retains the ability to participate in a biochemical reaction which it normally engages in its natural context.

One skilled in the art having read the present disclosure can readily recognize suitable in vitro assay conditions necessary to identify a molecule that can modulate binding of a transcription factor to a SNCA genomic variant. Typically, it will be an in vitro binding assay in which direct binding of a transcription factor to an immobilized oligonucleotide representing the SNCA genomic variant is measured. Exemplary suitable in vitro assays include, but are not limited to, a surface plasmon resonance and an electrophoretic mobility shift assay (EMSA). Briefly, in EMSA a radioactively labeled SNCA genomic variant oligonucleotide including but not limited to an oligonucleotide with SEQ ID NO:1, SEQ ID NO:2, SEQ ID NO:3, SEQ ID NO:4 or SEQ ID NO:5 is incubated with a recombinant transcription factor and a molecule to be tested as being a possible lead drug candidate. The ability of the molecule to modulate binding of the recombinant transcription factor to the labeled SNCA genomic variant oligonucleotide is then analyzed by gel electrophoresis. If the transcription factor is bound to the labeled SNCA genomic variant oligonucleotide (that is the molecule did not significantly modulate binding), the observed mobility of the labeled SNCA genomic variant oligonucleotide is shifted towards higher molecular weight.

The invention provides methods to identify a lead candidate for drug development for treatment of PD in carriers of a SNCA genomic variant involving whole cells. In some instances, the whole cells carry a SNCA genomic variant. The assay may contain cell extracts or other cellular components. The assay may also comprise a recombinant SNCA-mediated gene expression product wherein the gene or gene expression product retains the ability to participate in a biochemical or cellular reaction which it normally engages in its natural context.

Yet in another embodiment of the invention, the gene whose expression is mediated by a SNCA genomic variant includes, but is not limited to KLHL8, PTPN13, HSD17B13, HSD17B11, NUDT9, SPARCL1, DMP1, IBSP, MEPE, SPP1, PKD2, ABCG2, PPM1K, HERC6, HERC5, PIGY, PYURF, HERC3, NAP1L5, FAM13A, TIGD2, GPRIN3, SNCA-AS1, MMRN1, CCSER1, GRID2, PDZRN4, or a combination thereof. In a further embodiment, the gene whose expression is mediated by a SNCA genomic variant comprises HERC5, HERC6, SPARCL1, MMRN1, PDZRN4, or a combination thereof.

One specific embodiment of the invention provides a molecule that is capable of modulating the expression of a SNCA-mediated gene by modulating a transcription factor. As used herein, the term “modulating a transcription factor” includes inhibiting binding of the transcription factor to the SNCA genomic variant, inhibiting production of the transcription factor, e.g., by inhibiting transcription of the transcription factor gene and/or by inhibiting translation of mRNA that encodes the transcription factor.

The molecule used for modulating SNCA-mediated expression of a gene by inhibiting a transcription factor includes, but is not limited to, small molecules, oligonucleotides (including short interfering RNAs, RNAs and aptamers), peptides, polypeptides (including aptamers, zinc fingers and fragments thereof), proteins (including antibodies and fragments thereof) as well as derivatives or modified forms thereof. In some embodiments, the oligonucleotide is a subsequence or a segment of SEQ ID NO:1, SEQ ID NO:2, SEQ ID NO:3, SEQ ID NO:4 or SEQ ID NO:5. In particular, the oligonucleotide is 11 to 30 nucleotides in length comprising a consecutive nucleotide a subsequence or a segment of SEQ ID NO:1, SEQ ID NO:2, SEQ ID NO:3, SEQ ID NO:4 or SEQ ID NO:5. In one instance, the oligonucleotide is a single-strand oligonucleotide. Yet in another instance, the oligonucleotide is a double-strand oligonucleotide.

In another embodiment of the invention, a molecule is an oligonucleotide that can bind to the SNCA genomic variant that binds to the transcription factor. In some instances, the oligonucleotide is substantially complementary to the SNCA genomic variant that binds to the transcription factor. The term “substantially complementary to” or “substantially the sequence” refers to sequences which hybridize to the sequences provided under stringent conditions and/or sequences having sufficient homology with SNCA genomic variant that binds to the transcription factor such that the oligonucleotides of the invention hybridize to the SNCA genomic variant sequence. “Substantially” the same as it refers to oligonucleotide sequences also refers to the functional ability to hybridize or anneal with sufficient specificity to distinguish between the presence or absence of the variant. This is measurable by the temperature of melting being sufficiently different to permit easy identification of whether the oligonucleotide is binding to the SNCA genomic variant that binds to transcription factor. Unless the context requires otherwise, the term “SNCA genomic variant that binds to transcription factor” refers to a portion of the SNCA genomic variant gene that binds to the transcription factor.

Yet in some embodiments, the oligonucleotide is a phosphorothioate oligonucleotide, a phosphoramidite oligonucleotide, a methylphosphonate oligonucleotide, a locked-nucleic acid-modified oligonucleotide, a peptide nucleic acid oligonucleotide, or combinations thereof.

In another embodiment, the oligonucleotide is fused to a cell penetrating peptide.

In some embodiments, the molecule is a polypeptide or a protein that is a fragment of the transcription factor protein that can bind to the SNCA genomic variant. In a preferred embodiment, said peptide lacks the transactivation domain of the transcription factor.

In some embodiments the molecule is a zinc finger protein, an antibody or a fragment thereof that can bind to the SNCA genomic variant.

In a further embodiment in which the molecule is a polypeptide or a protein and fragments thereof the molecule is fused to a cell penetrating peptide to facilitate intracellular delivery of the molecule. In another embodiment the cell penetrating peptide has the amino acid sequence GRKKRRQRRRPQ. In another embodiment, the molecule is encoded by an expression vector. In a further embodiment the expression vector is a component of a viral gene delivery system.

The molecule can further include a pharmaceutically acceptable carrier. The terms “pharmaceutically acceptable carrier” and “pharmaceutically acceptable excipient” are used interchangeably herein and refer to an excipient that is useful in preparing a pharmaceutical composition that is generally safe, non-toxic and neither biologically nor otherwise undesirable, and includes excipient that is acceptable for human pharmaceutical use.

Still in other embodiments, the cell comprises a neuronal cell. Neuronal cells can be neuronal progenitor cells as well as differentiated neurons. Methods of the invention are applicable to a neuronal progenitor cell, a differentiated neuron or a combination thereof.

Methods of the invention include molecules that can inhibit SNCA-mediated gene expression by binding to the SNCA genomic variant, the transcription factor, or both.

Another aspect of the invention provides a method for identifying a molecule that can modulate the binding of a transcription factor to the SNCA genomic variant. This method can be used to identify a lead candidate for drug development for treatment of PD. The method typically involves an in vitro or a biochemical assay that does not contain whole cells. The assay may contain cell extracts or other cellular components. The assay may also comprise a substantially purified gene or gene expression product. In this context “substantially purified” refers to a composition where the gene or gene expression product is present at least 10-fold higher than in any naturally occurring context, and wherein elements of the natural context have been removed, yet wherein the gene or gene expression product retains the ability to participate in a biochemical reaction which it normally engages in its natural context. One skilled in the art having read the present disclosure can readily recognize suitable in vitro assay conditions necessary to identify a molecule that can modulate binding of a transcription factor to a SNCA genomic variant. Typically, it will be an in vitro binding assay in which direct binding of a transcription factor to an immobilized oligonucleotide representing the SNCA genomic variant is measured. Exemplary suitable in vitro assays include, but are not limited to, a surface plasmon resonance and an electrophoretic mobility shift assay (EMSA). Briefly, in EMSA a radioactively labeled SNCA genomic variant oligonucleotide is incubated with a recombinant transcription factor and a molecule to be tested as being a possible lead drug candidate. The ability of the molecule to modulate binding of the recombinant transcription factor to the labeled SNCA genomic variant oligonucleotide is then analyzed by gel electrophoresis. If the transcription factor is bound to the labeled SNCA genomic variant oligonucleotide, the observed mobility of the labeled SNCA genomic variant oligonucleotide is shifted towards higher molecular weight.

High-Throughput Screening Assays: Screening assays of the invention are designed to identify modulation of a function, activity or amount of an SNCA-mediated gene or gene expression product, e.g., the mRNA or the protein generated from the gene sequence. As used herein the term “modulation” means any change in activity of a function or amount of the transcribed gene, mRNA or protein, (together which are sometimes called the “target”) including any change in transcription rate or expression level, and includes inhibition or activation, and antagonist and agonist effects on the biochemical or biological activity of the target. Throughout this disclosure, the term “change” when referring to any biological activity, e.g., SNCA-mediated gene expression or activity of SNCA-mediated gene expression product, means the value is statistically different from a control (i.e., p<0.25, often p<0.1, and more often p<0.05). The term “control” of gene expression or activity of a gene expression product refers to a standard level against which gene expression or the activity of the gene expression product, respectively, in cell is or can be compared. In some embodiments, the control can be cells carrying the SNCA wild-type allele, meaning the SNCA-mediated gene expression level and/or activity measured in cells carrying the SNCA genomic variant is compared to the expression level and/or activity in SNCA wild-type cells. This allows a determination based on the expression or biological activity against cells or subject with the SNCA wild-type.

This specification discloses diverse functions of SNCA-mediated genes, either known from the art or implied from proteins of the same class, that may be used to assess modulation. Some functions may be assessed directly, such as the catalyzing of a specific reaction, or less directly, such as by measuring the accumulation of a downstream product. Many assay designs are available to those skilled in the art. Preferred assays are optimized for speed, efficiency, signal detection and low reagent consumption. (Zhang et al. (1999) J. Biomolec. Screen. 4(2):67). Assays can be reporter assays measuring gene transcription, gene translation or a biological activity of the gene expression product. Assays can be developed in a neuronal cell, a neuronal progenitor cell, a differentiated neuron, an oligodendrocyte, a fibroblast, a human embryonic kidney cell or another cell commonly used in drug discovery and development efforts known to one skilled in the art. The examples below include description of assays for accumulation of phospho-Ser473-Akt protein or Notch protein, which are examples of downstream products which can be measured in cells to identify compounds which modulate the expression or activity of one or more of the SNCA-mediated gene products. Based on the invention herein, those of skill in the art can now predictably develop screening assays for potential PD therapeutic agents based on assays designed to measure if the compound modulates the protein expression or functions disclosed herein, and other functions known or to be discovered now that their significance is understood.

In some embodiments, the screening assays of the invention, either cellular, cell extract or biochemically based with substantially purified genes or gene expression products, are designed for testing a plurality of compounds (e.g., millions) through high-throughput screening of chemical libraries.

Chemical libraries of test compounds that may be screened to identify a modulator can be obtained from numerous available resources or using any of the numerous approaches in library synthesis methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145). See also Dolle et al. (2010) Comprehensive Survey of Chemical Libraries for Drug Discovery and Chemical Biology: 2009. J. Comb. Chem., 2010, 12 (6), pp 765-806.

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Test compounds which successfully modulate the activity of an SNCA-mediated gene or expression product are attractive candidates for further investigation and secondary screening in alternative assays for potential use in treating PD. Compounds are considered “potentially useful for treatment” when first identified in a screening assay, because it is well known that initial successful hits rarely contain all the required features for a successful pharmaceutical. They are however extremely useful to allow researchers to identify a chemical core structure shared among compounds that effectively modulates or inhibits the target activity. Typically, when a core structure is identified, an extensive library of possibly thousands of related compounds is further developed with the aim of identifying a lead compound that meets all the criteria for a successful pharmaceutical candidate. The assay is used repeatedly through many rounds of screening of up to millions of compounds to ultimately identify a small group of lead compounds, one of which may eventually become an approved therapeutic agent.

In some embodiments of the invention, a possible lead molecule for treatment of PD identified by methods of the invention has 50% inhibition or activation concentration (IC₅₀ or EC₅₀) of about 500 μM or less, typically about 100 μM or less, often about 50 μM or less, more often about 10 μM or less, and most often about 500 nM or less.

Still another aspect of the invention provides a method for modulating SNCA-mediated expression of a SNCA-mediated gene in a cell. It should be appreciated that throughout this disclosure the term “SNCA-mediated expression of a gene” refers to expression of a gene caused by, due to, or mediated by a SNCA genomic variant. The SNCA-mediated expression of a gene can result in an increase or a decrease of the expression level of said gene. Such a method includes contacting a cell that is expressing or is capable of expression a gene mediated by a SNCA genomic variant with a molecule that is capable of modulating (i.e., increasing or decreasing) the expression of said gene. Modulation of expression of said gene means an increase or a decrease of the level of the expression product of said gene. In one embodiment, the SNCA-mediated gene whose expression is modulated by a molecule in a cell is located on human chromosome 4 within about a 2 Mb window upstream or downstream of the location of SNCA (ENSG00000145335; chromosomal location of SNCA 4:89724099-89838315). In another embodiment, the SNCA-mediated gene whose expression is modulated by a molecule in a cell is located on human chromosome 12. Exemplary SNCA-mediated genes whose expression is modulated by a molecule in a cell include, but are not limited to KLHL8, PTPN13, HSD17B13, HSD17B111, NUDT9, SPARCL1, DMP1, IBSP, MEPE, SPP1, PKD2, ABCG2, PPM1K, HERC6, HERC5, PIGY, PYURF, HERC3, NAP1L5, FAM13A, TIGD2, GPRIN3, SNCA-AS1, MMRN1, CCSER1, GRID2 and PDZRN4. In a further embodiment, the SNCA-mediated gene whose expression is modulated by a molecule in a cell comprises ABCG2, PPM1K, HERC3, HERC5, HERC6, SPARCL1, MMRN1, PDZRN4, or a combination thereof. In yet a further embodiment, the SNCA-mediated gene whose expression is modulated by a molecule in a cell comprises HERC5, HERC6, SPARCL1, MMRN1, PDZRN4, or a combination thereof.

Still another aspect of the invention provides a method for modulating the activity of a SNCA-mediated gene in a cell. Such a method includes contacting a cell that is expressing or is capable of expression a gene mediated by a SNCA genomic variant with a molecule that is capable of modulating (i.e., increasing or decreasing) the activity of a gene expression product of the gene. In one embodiment, the SNCA-mediated gene whose activity is modulated by a molecule in a cell is located on human chromosome 4 within about a 2 Mb window upstream or downstream of the location of SNCA (ENSG00000145335; chromosomal location of SNCA 4:89724099-89838315). In another embodiment, the SNCA-mediated gene whose activity is modulated by a molecule in a cell is located on human chromosome 12. Exemplary SNCA-mediated gene whose activity is modulated by a molecule in a cell include, but are not limited to KLHL8, PTPN13, HSD17B13, HSD17B11, NUDT9, SPARCL1, DMP1, IBSP, MEPE, SPP1, PKD2, ABCG2, PPM1K, HERC6, HERC5, PIGY, PYURF, HERC3, NAP1L5, FAM13A, TIGD2, GPRIN3, SNCA-AS1, MMRN1, CCSER1, GRID2 and PDZRN4. In a further embodiment, the SNCA-mediated gene whose activity is modulated by a molecule in a cell comprises ABCG2, PPM1K, HERC3, HERC5, HERC6, SPARCL1, MMRN1, PDZRN4, or a combination thereof. In yet a further embodiment, the SNCA-mediated gene whose activity is modulated by a molecule in a cell comprises HERC5, HERC6, SPARCL1, MMRN1, PDZRN4, or a combination thereof.

Clinical use of methods of the invention includes a method for treating a subject carrying a SNCA genomic variant suffering from PD. Methods of the invention include determining the SNCA genotype present in said subject; and (a) if said subject carries an allele of a SNCA variant, administering the subject with a molecule that is capable of modulating the binding of a transcription factor to the SNCA genomic variant, capable of modulating the expression of the gene or capable of modulating the activity of a gene expression product; or (b) if the subject does not carry a SNCA variant allele, administering the subject with a different molecule. In some embodiments, the step of determining the SNCA variant genotype in the subject comprises a step of determining whether the subject is homozygous or heterozygous for the SNCA variant.

The molecule of the invention can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, the molecule of the invention may be incorporated with excipient and used in the form of ingestible tablets, buccal tablets, troches, capsules, elixirs, suspensions, syrups, wafers, and the like. Such compositions and preparation can contain at least 0.10% of molecule of the invention. The percentage of the compositions and preparation can, of course, be varied and can conveniently be between about 1 to about 10% of the weight of the unit. The amount of molecule of the invention in such therapeutically useful compositions is such that a suitable dosage will be obtained. Preferred compositions or preparations according to the present invention are prepared such that an oral dosage unit form contains from about 1 to about 1000 mg of molecule of the invention.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the molecule of the invention, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the molecule of the invention can be incorporated into sustained-release preparations and formulation.

The molecule of the invention can also be administered parenterally. Solutions of the molecule of the invention as a free base or pharmacologically acceptable salt can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions of agents delaying absorption, e.g., aluminum monostearate and gelatin.

Sterile injectable solutions are prepared by incorporating the molecule of the invention in the required amount in the appropriate solvent with various other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

The physician will determine the dosage of the molecule of the invention which will be most suitable for prophylaxis or treatment and it will vary with the form of administration and the particular molecule chosen, and also, it will vary with the particular patient under treatment. The physician will generally wish to initiate treatment with small dosages by small increments until the optimum effect under the circumstances is reached. The therapeutic dosage can generally be from about 0.1 to about 1000 mg/day, and preferably from about 10 to about 100 mg/day, or from about 0.1 to about 50 mg/Kg of body weight per day and preferably from about 0.1 to about 20 mg/Kg of body weight per day and can be administered in several different dosage units. Higher dosages, on the order of about 2× to about 4×, may be required for oral administration.

The molecule of the invention can also be administered directly to the brain using stereotactic surgery.

The molecule of the invention can also be delivered by a viral vector.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### Examples

Transcription factor recognition motifs in SNCA genomic variants: Genomic variants in the SNCA gene that are linked to PD were analyzed for presence or absence of transcription factor binding sites by using the JASPAR database of human transcription factor recognition motifs. This study allowed the determination of whether new transcription factor binding sites are created or existing ones abolished by the SNCA genomic variants linked to PD. The DNA sequence around each SNCA genomic variant (FIG. 2) was searched for transcription factor binding sites using binding profiles from the JASPAR CORE database of experimentally defined transcription factor binding sites for eukaryotes (http://jaspar.genereg.net/) using a relative profile score threshold cut-off of 85%. A transcription factor binding site was classified as “gained” or “de novo” in a SNCA genomic variant if it is found by the JASPAR screen using the 85% threshold cut-off value and it did not occur in the screen when the wild-type sequence was used in the query. Similarly, a transcription factor binding site was classified as “lost” if it was not found using the SNCA genomic variant sequence as query with the 85% threshold cut-off value. An increase in score means that the score was higher in the SNCA genomic variant compared to the SNCA wild-type sequence. A decrease in score means that the score was lower in the SNCA genomic variant compared to the SNCA wild-type sequence. A summary of the results is shown in Table 3.

The rs104893878 variant (A30P) creates several de novo binding sites. One is for ID4 (inhibitor of differentiation or inhibitor of DNA-binding 4) (FIG. 3A). ID4 is a member of a subfamily of helix-loop-helix (HLH) transcription factor. ID transcription factors contain the HLH-dimerization domain but lack the DNA-binding basic domain. Consequently, ID proteins inhibit transcriptional transactivation by heterodimerization with bHLH proteins. ID4 can bind and inhibit among others Hes1. Neural defects and premature differentiation were seen in mice lacking ID4.

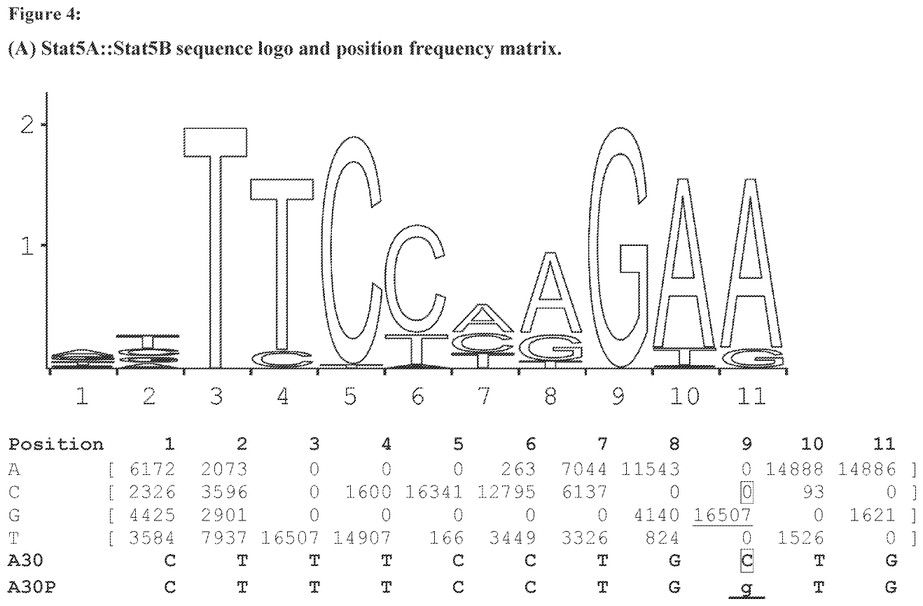

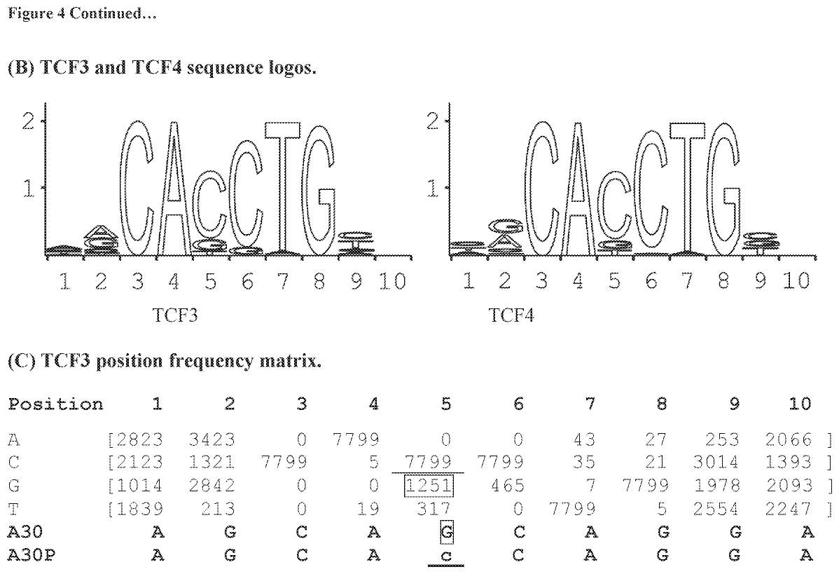

The rs104893878 variant also creates a de novo binding site for a Stat5a::Stat5b (Signal Transducer and Activator of Transcription 5a and 5b) heterodimer (FIG. 3A). In the brain STAT5A and STAT5B are activated by neuroprotective and neurotrophic erythropoietin and growth hormone^{xv}. This predicted binding site is located on the reverse strand. This novel binding motif has a score of 6.498, compared to 16.875 for the consensus binding motif, due to the presence of less frequent nucleotides in the last two positions (GTG instead of GAA; FIG. 4A).

The same variant also increases the scores for TCF3 and TCF4 binding motifs (Table 6, FIG. 4C), which are practically identical (FIG. 4B). TCF3 is required for B and T lymphocyte development but plays an important role in neurogenesis, as it is a repressor of the nuclear response to Wnt/β-catenin signaling, and protective during ischemia^{xvi}. This binding site has a comparatively low score, as the presence of an “A” in position 7 is rare. However, since a particular transcription factor may bind different motifs under certain conditions (e.g., potential competitive binding between Sox2 and TCF3^{xvii}), it can be concluded that this TCF3/4 binding motif plays a role in regulation of gene expression mediated by the SNCA genomic variant.

TABLE 6
TCF3 binding scores for A30, A30P and consensus sequence.

| | Transcription | | |
|---|---|---|---|
| Model ID | Factor | Score | Sequence |
| MA0522.2 | TCF3 | 13.569 | Consensus |
| MA0522.2 | TCF3 | 3.179 | A30 |
| MA0522.2 | TCF3 | 5.798 | A30P |

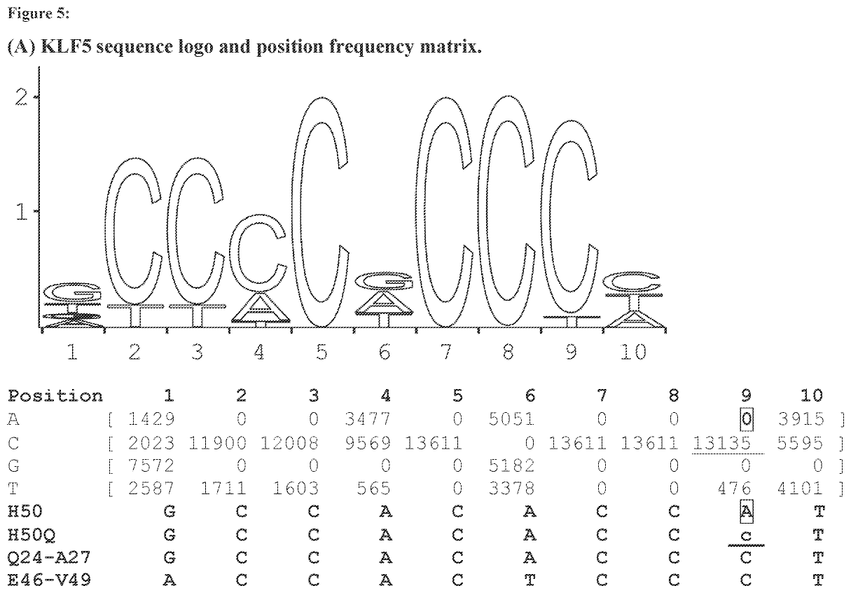

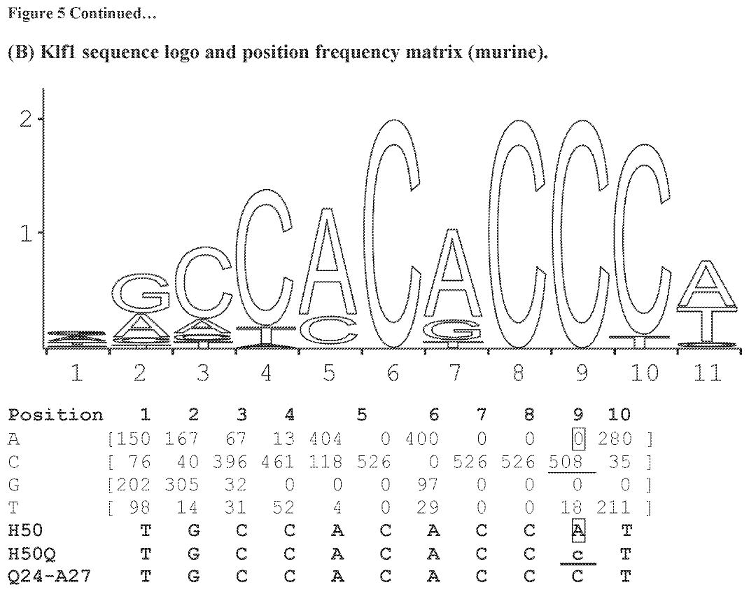

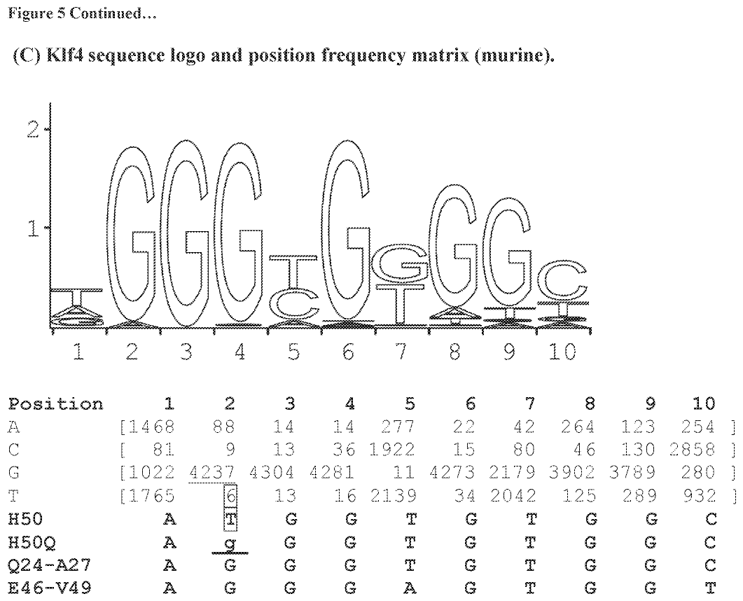

The rs201106962 (H50Q) genomic variant created a de novo KLF12 binding motif, with a high score (Table 3). It also increased the score of a KLF5 binding motif (Table 7) by changing a non-consensus A nucleotide with 0 appearances in the position weight matrix into a highly conserved, consensus matching C nucleotide with 13135 appearances in the human genome (FIG. 5A). H50Q increased the score of a Klf1 binding motif (Table 7), changing a non-consensus A nucleotide with 0 appearance in the position weight matrix into a highly conserved, consensus matching C nucleotide (FIG. 5B). In the same manner, it increased the score of a Klf4 binding motif (FIG. 5C, Table 7), changing a very rare “T” nucleotide in position 2 into a frequently found “G”. The JASPAR position weight matrix for Klf1 and Klf4 relates to the murine transcription factors. There is however no reported functional difference between mouse and human KLFs proteins. Krüppel-like factors (KLF) are a family of transcriptional regulators which function in a numerous of cellular processes. Most of them are expressed in neurons where they regulate neurite growth and axon regeneration^{xvii}. KLF5 is downregulated in the prefrontal cortex in schizophrenia patients, KLF4 is upregulated by NMDA or AMPA treatment in cortical neuron cultures.

TABLE 7
KLF5 and Klf1/4 binding scores for rs201106962 (H50Q) genomic
variant and wild-type (H50) and consensus sequences.

| | Transcription | | |
|---|---|---|---|
| Model ID | Factor | Score | Sequence |
| MA0599.1 | KLF5 | 15.466 | Consensus |
| MA0599.1 | KLF5 | 4.713 | H50 |
| MA0599.1 | KLF5 | 13.531 | H50Q |
| MA0493.1 | Klf1 | 17.059 | Consensus |
| MA0493.1 | Klf1 | 9.173 | H50 |
| MA0493.1 | Klf1 | 15.659 | H50Q |
| MA0039.2 | Klf4 | 15.509 | Consensus |
| MA0039.2 | Klf4 | 7.588 | H50 |
| MA0039.2 | Klf4 | 15.153 | H50Q |

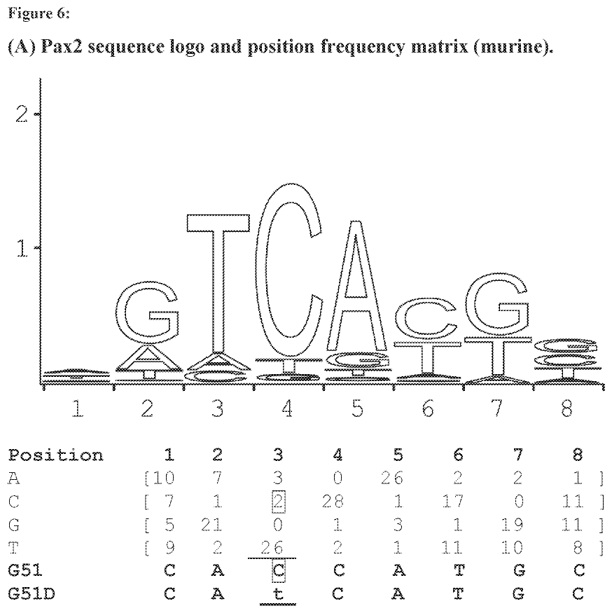

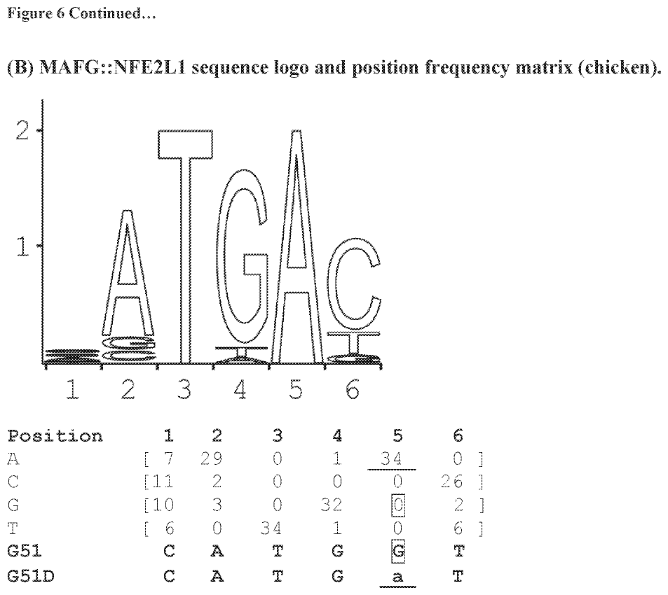

The rs431905511 (G51D) genomic variant causes the loss of Klf1, Klf4 and KLF5 sites observed in the SNCA wild-type. However, these were non-consensus sites with relatively low scores. This genomic variant however, leads to the creation of a de novo MAFG::NFE2L1 (heterodimer between MAFG and NFE2L1) (Table 3), which may impair binding to MAFG and/or NFE2L1 (decoy effect). Further this genomic variant creates a de novo site for Pax2 (FIG. 6A).

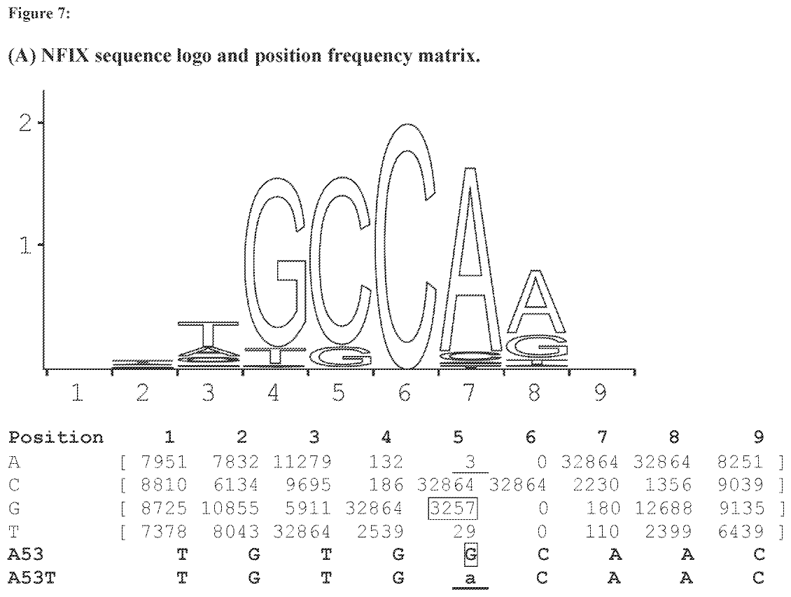

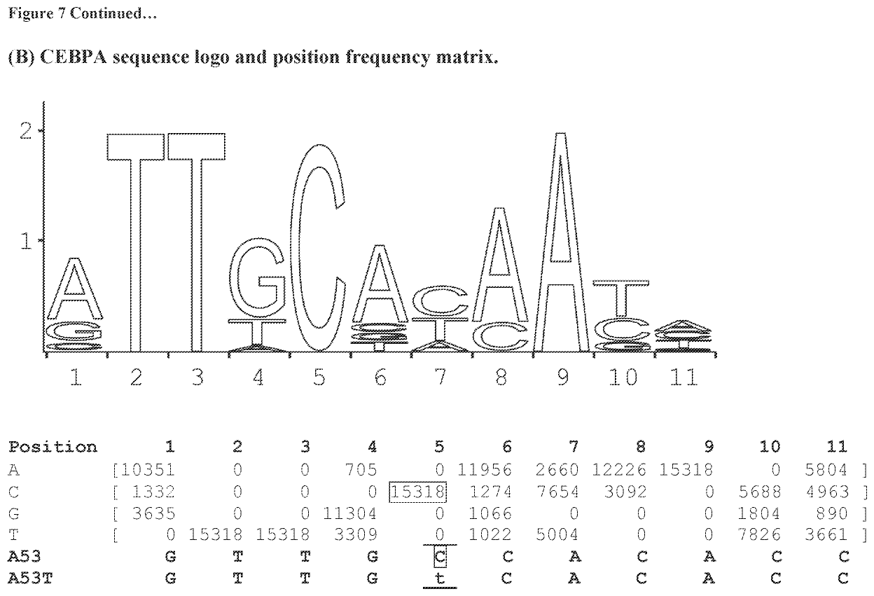

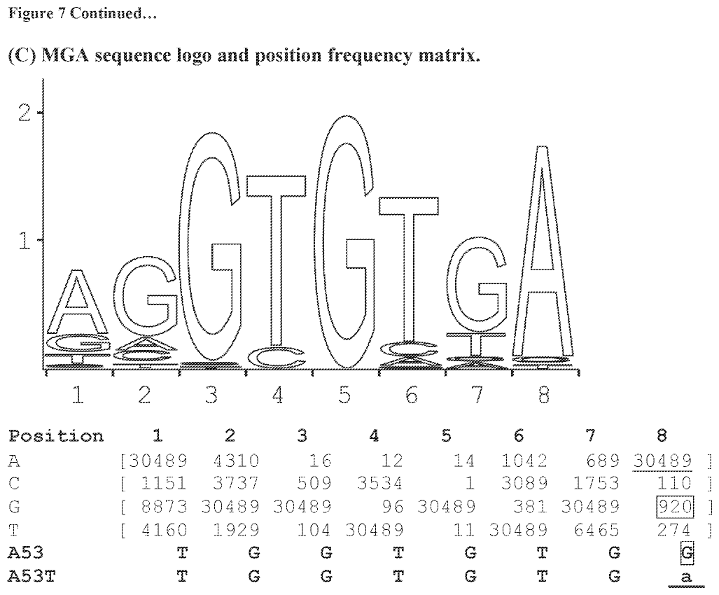

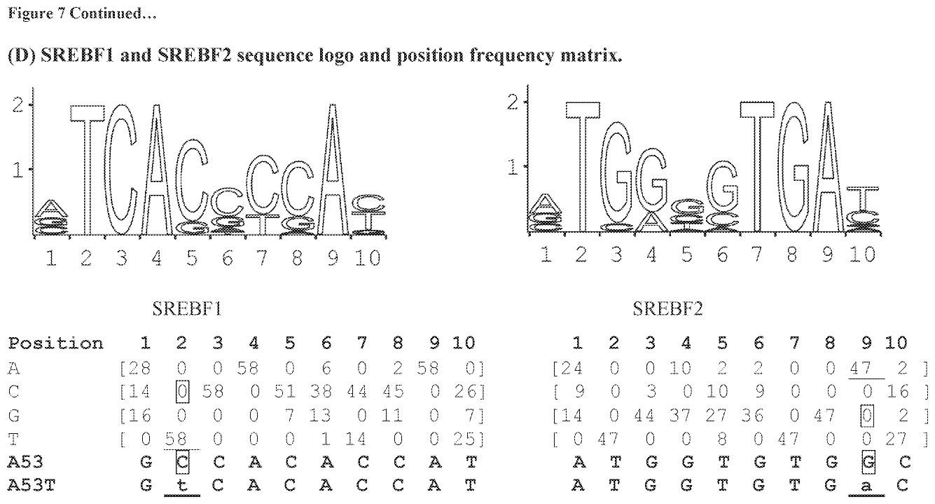

The rs104893877 (A53T) genomic variant causes the loss of the same Klf1, Klf4 and KLF5 as observed with rs431905511 (G51D). Importantly, it also negatively affects high scoring NFIX and CEBPA sites present in SNCA wild-type (FIGS. 7A and 7B). In the NFIX site a frequent G nucleotide is exchanged by the rarest nucleotide (A) at this position. In the CEBPA site the nucleotide at position 5 is always a C (15318 occurrences) in the human transcription factor motif database while in the rs104893877 variant the nucleotide at this position is changed to a T.

The rs104893877 (A53T) genomic variant also creates high scoring de novo motifs for Sterol Regulatory Element Binding Transcription Factor 1 and 2 (SREBF1 and SREBF2) (Table 3, FIG. 7), two antipsychotic-activated transcription factors controlling cellular lipogenesis (Table 5).

The rs104893877 (A53T) genomic variant further creates an MGA (MAX Dimerization Protein) recognition site (FIG. 7C). MGA is a dual-specificity transcription factor, regulating the expression of both MAX-network and T-box family target genes. MGA requires heterodimerization with Max for binding to the preferred Myc-Max-binding site^{xix}.

The rs104893875 (E46K) genomic variant, although in the same region as genomic variants associated with PD, leads to dementia with Lewy Bodies, as does the SNCA gene duplication in some instances. This invention noted that this variant does not affect the high scoring KLF sites but causes the loss of a high scoring E2F6 site. E2F6 is a transcriptional repressor of a subset of E2F-dependent cell cycle genes and other genes involved in development. E2F6 has been demonstrated to repress DNA damage-and hypoxia induced apoptosis. E2F6 is involved in the regulation of PPM1K in the SNCA genomic region.

From what was seen above several of transcription factors binding motifs affected by the SNCA mutations (Table 3) are also present in proximal regions of genes lying in the vicinity of the SNCA (Table 4). Some of these binding motifs may represent weak affinity binding sites (Stat5A::Stat5b), that could play a role as decoy or influence gene regulation in other ways.

Most SNCA genomic variants are anticipated to regulate several genes (Table 5) both in cis and in trans. For instance, rs104893878 (A30P) regulates any of the HERC5 and HERC6 E3 ligases, as well as protein phosphatase, Mg²⁺/Mn²⁺ dependent 1K (PPM1K), SPARCL1 and PDZRN4 on chromosome 12. Rs201106962 (H50Q) is anticipated to regulate ATOH1, HERC5, HERC6, ABCG2, PPM1K, MMRN1, SPARCL1 and PDZRN4 in trans. Rs431905511 (G51Q) is anticipated to regulate HERC3 as well as HERC5 and HERC6, ABCG2, PPM1K, MMRN1, SPARCL1 and PDZRN4 in trans. Finally, rs104893877 (A53T) is anticipated to affect expression of the same genes (HERC3, HERC5, HERC6, ABCG2, PPM1K, MMRN1, SPARCL1, PDZRN4).

ABCG2 and PPM1K cause mitochondrial dysfunction, and thus perturbate mTOR/Akt activation. HERC5 and HERC6 are engaged in PARK2 activation. Improper regulation of these E3 ligases either by gene repression as observed in A53T variant human neurons will cause mitochondrial dysfunction and SNCA aggregation in the substantia nigra (inhibition of proteasomal degradation and mitophagy). HERC3 may affect the Notch pathway and formation of Lewy bodies through its binding to ubiquilin-1. PDZRN4 is a regulator of Numb/Notch signaling, whose dysregulation affects the Notch pathway mTOR/Akt activation. SPARCL1 and MMRN1 (Multimerin 1) are soluble proteins involved in adhesion and synaptic function and plasticity. Notably, SPARCL1 affects dendrite formation and synapse formation, whereas MMRN1 binds to alphav/beta3, a type of integrin that has been involved in synaptic homeostasis. SPARCL1-SPARC (Secreted Protein Acidic And Cysteine Rich) expression ratio is crucial for synapse maintenance; overexpression of SPARCL1 as observed in A53T variant human neurons will lead to sustained synaptic retraction. Overexpression of multimerin 1 as detected in A53T variant human neurons will cause altered AMPAR endocytosis and synaptic scaling. Connections between synaptic protein homeostasis and the mechanisms of PD are particularly strong^{xx}.

These examples demonstrate that SNCA genomic variants have profound effects on the presence or absence of transcription factor recognition motifs.

As can be clearly seen, the SNCA genomic variants create de novo binding sites for transcription factors. By binding to the genomic location of SNCA variants and the proximal promoter region of one of the genes harboring transcription factor binding sites this transcription factor leads to inappropriate (increase or decrease) expression of SNCA-mediated genes. Thus, some aspects of the invention provide a method of treating PD by decreasing or increasing the expression of one or more SNCA-mediated genes or the activity of a gene expression product.

The present invention discloses an analysis of SNCA-mediated genes and determined that seven of these genes are directly linked to the Notch activation pathway, namely ABCG2, PPM1K, HERC3, HERC5, HERC6, SPARCL1 and PDZRN4. Other genes listed in Table 4 could also interfere with the Notch activation pathway.

For each of these genes, the invention describes an examination of the CHEA (ChIP Enrichment Analysis) Transcription Factor Targets dataset and ENCODE Transcription Factor Target dataset provided by the Harmonizome (http://amp.pharm.mssm.edu/Harmonizome/about), a publicly available collection of information and research findings about genes and proteins from 114 datasets collected from 66 online resources (e.g. genomics, transcriptomics, proteomics, metabolomics, from cells, tissues, model organisms, and patients). CHEA integrate the results of experiments such as ChIP-chip, ChIP-seq, ChIP-PET and DamID which profile the binding of transcription factors to DNA at a genome-wide scale and provide a list of the transcription factors binding the promoter of the gene of interest^{xxi}. The ENCODE Transcription Factor Targets compile transcription factor DNA-binding sites identified by ChIP-seq.

Notch pathway specific interference (SPARCL1): The SPARCL1 (SPARC Like 1) gene is located on chromosome 4, and lies 2.2 Mb upstream from the alpha-synuclein gene. The expression of SPARCL1 in PD neurons is regulated by the transcriptional enhancer activity of SNCA genomic variants. The SPARCL1 promoter has binding sites for C/EBPalpha. TCF3 has been identified as a SPARCL1 regulator by CHEA studies. The JASPAR analysis described in this invention has identified several high scoring binding motifs for MEIS1, Hic2 and NFIX and at least one high scoring binding motif for FOXD2, FOXO4/6, and MEIS3 in this gene's 5′ sequences. SPARC-like protein 1, also known as hevin, regulates cell-matrix interactions in the developing brain. SPARCL1 is an astrocyte secreted protein that can also be detected inside neurons^{xxii}. In vitro studies have shown that NFIX drives its expression in astrocyte. It is an important factor for functional synapse assembly^{xxiii}. In the adult brain, Notch signaling influences axon guidance, morphogenesis of dendritic arbors, long-term synaptic plasticity, memory and survival, mainly through its non-canonical pathway. In postmitotic neurons, Notch appears to be is enriched in the dendrites and cell soma, while ligand Jagged1 is enriched in axons. Moreover, Notch is likely to be stored in RNA-granules in the dendrite. Dysregulation of SPARCL1 affects dendrite formation and consequently proper Notch-ligand interaction and activation. These and other known biological activities of SPARCL1 can be employed by those skilled in the art to design screening assays to screen for test compounds that modulate these activities, thereby identifying potential therapeutic agents for the treatment of PD. Standard reporter assays incorporating the gene or gene expression product can also be employed.

Notch pathway specific interference (ABCG2): The ABCG2 (ATP binding cassette subfamily G member2) gene is located on chromosome 4, 1.6 Mb upstream of SNCA. The expression of ABCG2 in PD neurons is regulated by the transcriptional enhancer activity of SNCA genomic variants. ABCG2 belongs to the ATP-binding cassette superfamily of transmembrane proteins that play an important role in cancer multidrug resistance. It is expressed in several organs including the liver. ABCG2-deficient hepatocytes show disruption of mitochondrial dynamics and functions, due to elevating intracellular protoporphyrin IX, which leads to upregulation of DRP-1-mediated mitochondrial fission^{xxiv}. ABCG2 is also ubiquitously expressed in stem cells including those in the developing nervous system. ABCG2 was identified as a KLF1 and EOMES target gene in CHEA analysis. The JASPAR analysis described in this invention has detected several very high scoring binding motifs for MEIS1 and 3 and MAFG::NFE2L1 as well as least one high scoring binding motif for FOXD2, FOXO4 and FOXO6 and NFIX in the 5′ sequences of the ABCG2 gene. The ENCODE Transcription Factor Target dataset identifies ABCG2 as a ZNF143 target gene. A Notch-activated signaling cascade has been evidenced that interacts with mitochondrial remodeling proteins to regulate cell survival. In this pathway, PINK1 recruits Notch to mitochondria and then activates mechanistic target of rapamycin complex 2 (mTORC2)/Akt signaling to influence mitochondrial dynamics and function. Dysregulation of ABCG2 affects mitochondrial dynamics and thus impedes Pink1/mTORC2/Akt activation. These and other known biological activities of ABCG2 can be employed by those skilled in the art to design screening assays to screen for test compounds that modulate these activities, thereby identifying potential therapeutic agents for the treatment of PD. Standard reporter assays incorporating the gene or gene expression product can also be employed.

Notch pathway specific interference (PPMIK): The PPM1K (Protein phosphatase, Mg²⁺/Mn²⁺ dependent 1K) gene is located on chromosome 4, upstream of SNCA. The expression of PPM1K in PD neurons is regulated by the transcriptional enhancer activity of SNCA genomic variants. PPM1K is a Mn²⁺/Mg²⁺-dependent protein phosphatase that is involved in the regulation of the mitochondrial permeability transition pore (MPTP), a large conductance uncoupling channel that opens in the inner mitochondrial membrane often in pathological conditions^{xxv}. MPTP is also regulated by PINK1^{xxvi}. PPM1K is regulated by numerous transcription factors, notably SREBF2 and STAT5A (CHEA/ChIP Enrichment Analysis). The JASPAR analysis described in this invention has identified several very high scoring binding motifs for FOXD2, Klf4, KLF5, MAFG::NFE2L1, MEIS1/3 and NFIX and at least one high scoring binding motif for E2F6, FOXO4/6, and HIC2 in the 5′ sequences of the PPM1K gene. Non-canonical Notch pathway regulates cell survival through activation of mechanistic target of rapamycin complex 2 (mTORC2)/Akt signaling. Dysregulation of PPM1K affects mitochondrial permeability and thus impedes Akt/Pink1/mTORC2 activation. These and other known biological activities of PPM1K can be employed by those skilled in the art to design screening assays to screen for test compounds that modulate these activities, thereby identifying potential therapeutic agents for the treatment of PD. Standard reporter assays incorporating the gene or gene expression product can also be employed.

Notch pathway specific interference (HERC3, HERC5, HERC6): The HERC3, HERC5 and HERC6 genes lie 1.35, 1.27 and 1.2 Mb, respectively, upstream from SNCA on chromosome 4. The expression of HERC3, HERC5 and HERC6 in PD neurons is regulated by the transcriptional enhancer activity of SNCA genomic variants. HERC3 (HECT and RLD domain containing E3 ubiquitin protein ligase 3), HERC5 (HECT and RLD domain containing E3 ubiquitin protein ligase 5) and HERC6 (HECT and RLD domain containing E3 ubiquitin protein ligase 6) are members of the so called small HERC protein family and exhibit a high degree of homology. All three are E3 ubiquitin-protein ligases, interact with each other, localize to the cytosol or the same cellular structures (late endosomes and lysosomes) and are involved in vesicular trafficking. HERC3 is controlled by a long list of transcription factors. HERC3 proximal enhancer harbors 47 transcription factors binding motifs and notably STAT5A and TCF3 (CHEA Transcription Factor Targets dataset). The JASPAR study of the present invention found several very high scoring binding motifs for FOXD2, FOXO4, FOXO6, MAFG::NFE2L1 and MEIS1 and 3 in its 5′ DNA sequence. HERC5 expression is regulated by several transcription factors. The JASPAR analysis described in this invention has found several high scoring binding motifs for KLF4 and 5, NFIX and SP3 and at least one high scoring binding motif for TCF4, Pax2, Hic2 and E2F6 within this gene 5′ DNA sequence. Moreover, KLF5 negatively regulates the expression of HERC5 in some instances^{xxvii}. HERC6 is regulated by several transcription factors as well. JASPAR analysis has found high scoring binding sites for ID4, FOXD2, FOXO4 and 6, HIC2, MEIS1 and 2, NFIX, SREBF2, TBX4/5, TCF3/4 and MGA within this gene 5′ DNA sequence. HERC3 binds and regulates ubiquilin-1 and 2. Ubiquilin-1 binds the autophagosome marker LC3, and regulates autophagosome formation. LC3 and other autophagic markers can be found in Lewy bodies inclusions. HERC5 is the major E3 ligase for ISG15 conjugation^{xxvii}. ISG15, a ubiquitin-like protein, is a key type I interferon effector. Recently, parkin was identified as a novel target of ISGylation specifically mediated by HERC5. The ISGylation of two sites, Lys-349 and Lys-369, situated in the in-between-ring domain of parkin promotes parkin's ubiquitin E3 ligase activity by suppressing the intramolecular autoinhibited conformation^{xxix}. What's more, Type I interferon induced expression of ISG15 leads to HERC5 ISGylation of Beclin-1, that performs an important role in negative regulation of autophagy. HERC6 also plays a role in protein ISGylation in vivo^{xxx}. Notch is continuously sorted to other endocytic compartments to prevent inappropriate signaling. Non-canonical Notch signaling cascade interacts with mitochondrial remodeling proteins to regulate cell survival through PINK1/mTORC2/Akt activation. After Notch activation, PARK2 is recruited by PINK1 to the membrane of depolarized mitochondria where it ubiquitinates mitofusins, in so doing allowing autophagic clearance of the damaged organelle. Concomitantly and to circumvent sustained activation, Notch is degraded by the E3 ligase FBXW7. Dysregulation of HERC3 affects vesicular trafficking, and binding with ubiquilin 1 and ubiquilin-1 function, affecting both Notch recycling and degradation, which in turn impedes PINK1/mTORC2/Akt activation. Dysregulation of HERC5 and HERC6 affects PARK2 activation through trigger induced ISGylation, and subsequently PARK2 recruitment to the mitochondria, which in turn impedes PINK1/mTORC2/Akt activation. These and other known biological activities of HERC3/5/6 can be employed by those skilled in the art to design screening assays to screen for test compounds that modulate these activities, thereby identifying potential therapeutic agents for the treatment of PD. Standard reporter assays incorporating the gene or gene expression product can also be employed.

Notch pathway specific interference (PDZRN4): The PDZRN4 gene (PDZ Domain Containing Ring Finger 4, gene ID ENSG00000165966) is located on chromosome 12, and lies about 1 Mb downstream from LRRK2. The expression of PDZRN4 in PD neurons is regulated by the transcriptional enhancer activity of SNCA genomic variants. PDZRN4 is a member of the LNX (Ligand of Numb Protein-X) family. LNX members typically contain an amino-terminal RING domain adjacent to either two or four PDZ domains. PDZ-containing scaffold proteins can bind PDZ-binding motifs such as the ones found at the C-terminus of DLL1, DLL4 and JAG11 ligands. They are therefore expected to play an important role in the Notch signaling. LNX proteins are E3 ubiquitin ligases. LNX proteins mediate the NUMB-dependent degradation of Notch. Therefore, PDZRN4, a member of the LNX E3 ubiquitin ligases can mediate the NUMB-dependent degradation of Notch. PDZRN4 5′region contains binding motifs for transcription factors HIC2, ID4, MGA, NFIX, TBX1, TBX15, TBX4/5 and TCF3/4. PDZRN4 has been described as a target gene of NFIX in the hippocampus. The present invention discloses a binding site for EOMES and TCF4 in the promoter of the PDZRN4 gene (CHEA Transcription Factor Targets dataset). A ZNF143 binding was identified near the transcription start site of PDZRN4 (ENCODE Transcription Factor Target dataset). For instance, the SNCA genomic variant rs104893878 (A30P) affects the recognition motif for ID4, TCF3 and TCF4. In another instance, the SNCA genomic variant rs201106962 (H50Q) affects recognition motifs for MGA, TBX4 and 5 and HIC2. In yet another instance, the SNCA genomic variant rs431905511 (G51D) affects recognition motifs for MGA, TBX4 and TBX5. The SNCA genomic variant rs104893877 (A53T) affects recognition motifs for EOMES, TBX1, TBX15, ZNF143, MGA, TBX4, TBX5, and NFIX.

Determination of expression levels of SNCA-mediated genes in cells: Human induced pluripotent stem cells (iPSC) with a known genotype of SNCA genomic variants are cultivated and differentiated into neurons. These cells can be derived from a subject carrying a SNCA genomic variant with PD or from a healthy subject carrying a SNCA genomic variant. These cells can also be derived from cells carrying the SNCA wild-type allele by introducing a SNCA genomic variant using molecular biology methods know to one skilled in the art including but not limited to CRISPR/CAS9 technology. There are numerous protocols available to the one skilled in the art to perform cultivation of iPSC and achieve differentiation into neurons, for example to dopaminergic neurons. Cells are typically received in cryovials, cells are then thawed and seeded in an appropriate culture vessel at a density of approximately 50,000 cells/cm². Cells are then grown in the presence of a neuronal differentiation medium for several days, typically three days, and cultivated for a further several weeks in the presence of a neuronal maintenance medium. Cells are assessed several weeks after differentiation for neuronal and synaptic marker expression, typically after 35 days. Neuronal markers are typically Tuj1 and MAP2, while synaptic markers are PSD-95 (postsynaptic terminals) and synaptophysin (presynaptic terminals). A commonly used marker to demonstrate differentiation to dopaminergic neurons is tyrosine hydroxylase. Cell cultures containing a SNCA genomic variant or the SNCA wild-type gene are then analyzed for the expression of SNAC-mediated genes. From all the neuronal cultures the media are removed and stored under appropriate conditions known to the skilled artisan. The neurons are then rinsed with an appropriate buffer, for example phosphate-buffered saline, and lysed. The lysate is then analyzed for presence of the expressed protein encoded by the SNCA-mediated gene using one of the numerous methods available to the one skilled in the art. Cell lysates are prepared and mixed with an appropriate loading buffer. The levels of specific proteins in the lysate are determined by a combination of sodium dodecyl sulfate polyacrylamide gel electrophoresis (SDS-PAGE) of the lysates with a specific detection method (Western blot) using antibodies directed to the proteins to be analyzed. The levels of KLHL8, PTPN13, HSD17B13, HSD17B11, NUDT9, SPARCL1, DMP1, IBSP, MEPE, SPP1, PKD2, ABCG2, PPM1K, HERC6, HERC5, PIGY, PYURF, HERC3, NAP1L5, FAM13A, TIGD2, GPRIN3, SNCA-AS1, MMRN1, CCSER1, GRID2, PDZRN4 or any of the genes listed in Table 4 in neurons with a SNCA genomic variant are compared to levels in neurons with a SNCA wild-type allele. The expression levels of the SNCA-mediated gene are also determined by quantification of messenger RNA (mRNA) levels using quantitative polymerase chain reaction (qPCR) and compared in neuronal cells carrying a SNCA genomic variant versus the SNCA wild-type allele. Expression levels of some of the SNCA-mediated genes can also be measured in the media collected from neuronal cell cultures and compared between media from SNCA genomic variant bearing neurons and SNCA wild-type allele bearing neurons.

In order to assess the activity of a molecule or a pharmaceutical composition on the expression of a SNCA-mediated gene the same cell culture conditions are employed. However, in this case the cell culture is incubated for several days, typically two days, most typically for twelve to 24 hours with a pharmaceutical composition before medium collection and lysate processing. In some instances, shorter incubation times lasting hours or minutes can be employed. The pharmaceutical composition can contain a molecule selected from, but not limited to, small molecules, oligonucleotides (including short interfering RNAs, RNAs and aptamers), peptides, polypeptides (including aptamers, zinc fingers and fragments thereof), proteins (including antibodies and fragments thereof) as well as derivatives or modified forms thereof.

It is readily apparent to one skilled in the art that this described method is not limited to neuronal cultures but can be applied to a neuronal cell line, neuronal progenitor cells, oligodendrocytes, cells collected from a patient with PD, who is a carrier of a SNCA genomic variant, or cells derived from cells collected from a human subject such as induced pluripotent stem cells derived from fibroblasts or plasma cell. It is readily apparent that this assay can be performed using whole blood, blood plasma, blood serum, sputum, saliva, urine, lymph or cerebrospinal fluid collected from human subjects carrying a SNCA genomic variant, and can be used to diagnose PD in these subjects. It is further apparent to one skilled in the art that this assay can be performed with human embryonic kidney cells, fibroblasts or other cells commonly used in drug discovery and development.

Determination of the activity of SNCA-mediated genes in a cellular assay: Differentiated neurons derived from human iPSC, neuronal progenitor cells, cells of a neuronal cell line such as PC12 or SH-SY5Y are maintained in culture. Specifically, human dopaminergic neurons with the SNCA A53T and the SNCA wt genotype are cultured. Alternatively, human dopaminergic neurons with other SNCA genomic variants and the SNCA wt genotype are cultured.

Levels of both total and active phospho-Ser⁴⁷³-Akt or levels of Notch are determined in the lysate of cultured cells by a combination of sodium dodecyl sulfate polyacrylamide gel electrophoresis (SDS-PAGE) of the lysates with a specific detection method using antibodies directed to total and active phospho-Ser⁴⁷³-Akt or Notch, using Western blots. The one skilled in the art knows of a multitude of alternative methods to determine protein levels in cell lysates.

The activity of a molecule on modulation of the SNCA-mediated gene expression or the activity of a gene expression product is measured by the levels of total and active phospho-Ser⁴⁷³-Akt or Notch in cells with a SNCA genomic variant that were treated with vehicle containing said molecule and compared to the levels in control cells treated with vehicle not containing said molecule. The control cells can carry the SNCA wild-type allele or a SNCA genomic variant. Typically, the cell culture is incubated for 1 to 48 hours with the molecule. The molecule used for modulating the expression or activity of a SNCA-mediated gene includes, but is not limited to, small molecules, oligonucleotides (including short interfering RNAs, RNAs and aptamers), peptides, polypeptides (including aptamers, zinc fingers and fragments thereof), proteins (including antibodies and fragments thereof) as well as derivatives or modified forms thereof.

It is readily apparent to one skilled in the art that this described method can be applied to a neuronal cell line, neuronal progenitor cells, oligodendrocytes, cells collected from a patient with PD who is a carrier of a SNCA genomic variant, or cells derived from cells collected from a human subject such as induced pluripotent stem cells derived from fibroblasts or plasma cells. It is readily apparent that this assay can be performed using whole blood, blood plasma, blood serum, sputum, saliva, urine, lymph or cerebrospinal fluid collected from human subjects carrying a SNCA genomic variant, and can be used to diagnose PD in these subjects. It is further apparent to one skilled in the art that this assay can be performed with human embryonic kidney cells, fibroblasts or other cells commonly used in drug discovery and development.

Determination of the activity of SNCA-mediated genes in a biochemical assay: A method for measuring modulation by a molecule of the activity of a SNCA-mediated gene expression product can comprise a biochemical assay. A biochemical assay is substantially devoid of whole cells but may contain cell extracts or other cellular components.

On aspect of measuring the activity of a SNCA-mediated gene expression product in a biochemical assay includes measuring the binding of a molecule to the SNCA-mediated gene expression product. The SNCA-mediated gene expression product can be immobilized on a solid support and the molecule is brought into contact with it in a reaction chamber. The binding of the molecule is quantified in an appropriate instrument using techniques known to one skilled in the art. In a preferred embodiment, the binding is quantified by surface plasmon resonance technology. Alternatively, the molecule is immobilized to the solid support and the SNCA-mediated gene expression product is brought into proximity in an appropriate reaction chamber. The SNCA-mediated gene expression product can be a recombinantly expressed, or a fragment thereof.

Another aspect of measuring the activity of a SNCA-mediated gene or activity of a gene expression product in a biochemical assay includes measuring the outcome of modulating the activity of a SNCA-mediated gene. A further aspect is measuring the activity of the E3 ubiquitin ligase PDZRN4. An E3 ubiquitin ligase recruits an E2 ubiquitin-conjugating enzyme and a protein substrate to assist in or directly catalyze the transfer of a ubiquitin from the E2 ubiquitin-conjugating enzyme to the protein substrate. In one embodiment, the E3 ubiquitin ligase activity of PDZRN4 is measured on its substrate Notch. In another embodiment the PDZRN4 activity on its substrate Notch is determined by the level of ubiquitination of Notch. In a further embodiment the E3 ubiquitin ligase activity assay of PDZRN4 includes the scaffolding protein NUMB. There are numerous assays known to one skilled in the art to measure the activity of E3 ubiquitin ligases on their substrates.

Determination of the activity of a molecule on transcription factor binding to a SNCA genomic variant: A method for measuring modulation of binding of a transcription factor having an effect on the expression of a SNCA-mediated gene in the presence of a molecule can comprise a biochemical assay. A biochemical assay is substantially devoid of whole cells but may contain cell extracts or other cellular components, comprises of a recombinant or chemically synthesized transcription factor, a recombinant or chemically synthesized oligonucleotide, and a detection method to measure the binding of the transcription factor to the oligonucleotide. There are numerous methods available to the ones skilled in the art to measure the binding of a transcription factor to an oligonucleotide.

The biochemical assay can comprise an electrophoretic mobility shift assay (EMSA). An oligonucleotide including but not limited to one containing the SEQ ID NO:1, SEQ ID NO:2, SEQ ID NO:3, SEQ ID NO:4 or SEQ ID NO:5 is labeled with a radioactive, fluorescent or biotin label and incubated with a transcription factor to allow complex formation. In one embodiment, the transcription factor or a fragment thereof is recombinantly expressed. In another embodiment, the transcription factor is chemically synthesized or recombinantly expressed ID4, Stat5a, Stat5b, TCF3, TCF4, Klf12, SP3, Klf1, Klf4, KLF5, TBX4, TBX5, Pax2, MAFG::NFE2L1, EOMES, FOXD2, FOXO6, Pax2, MEIS1, MEIS2, MEIS3, SREBF2 or MGA, or a fragment thereof. In another embodiment, ID4, Stat5a, Stat5b, TCF3, TCF4, Klf12, SP3, Klf1, Klf4, KLF5, TBX4, TBX5, Pax2, MAFG::NFE2L1, EOMES, FOXD2, FOXO6, Pax2, MEIS1, MEIS2, MEIS3, SREBF2 or MGA, or a fragment thereof, is contained in a nuclear extract of a cell. In one embodiment the oligonucleotide is 11 to 30 nucleotides in length comprising 11 to 30 consecutive nucleotides within SEQ ID NO:1, SEQ ID NO:2, SEQ ID NO:3, SEQ ID NO:4 or SEQ ID NO:5. The reaction mixture is then analyzed by gel electrophoresis. If ID4, Stat5a, Stat5b, TCF3, TCF4, Klf12, SP3, Klf1, Klf4, KLF5, TBX4, TBX5, Pax2, MAFG::NFE2L1, EOMES, FOXD2, FOXO6, Pax2, MEIS1, MEIS2, MEIS3, SREBF2, or MGA, or a fragment thereof is bound to the SCNA genomic variant oligonucleotide it shifts the observed mobility of the labeled oligonucleotide towards an apparent higher molecular weight.

In a further embodiment, the biochemical assays can comprise surface plasmon resonance. Instruments employing surface plasmon resonance are known to the one skilled in the art. In a typical surface plasmon resonance assay, an oligonucleotide including but not limited to one containing the SEQ ID NO:1, SEQ ID NO:2, SEQ ID NO:3, SEQ ID NO:4 or SEQ ID NO:5 is immobilized on a chip which is a component of the surface plasmon resonance detection instrument. In another embodiment the oligonucleotide is 11 to 30 nucleotides in length comprising 11 to 30 consecutive nucleotides within SEQ ID NO:1, SEQ ID NO:2, SEQ ID NO:3, SEQ ID NO:4 or SEQ ID NO:5. A solution containing a chemically synthesized or recombinantly expressed transcription factor ID4, Stat5a, Stat5b, TCF3, TCF4, Klf12, SP3, Klf1, Klf4, KLF5, TBX4, TBX5, Pax2, MAFG::NFE2L1, EOMES, FOXD2, FOXO6, Pax2, MEIS1, MEIS2, MEIS3, SREBF2, or MGA, or a fragment thereof, is contacted with the oligonucleotide immobilized on the chip and surface plasmon resonance is used to detect binding of the oligonucleotide to one of said transcription factors. In another embodiment, the same technology is applied but the chemically synthesized or recombinantly expressed transcription factor ID4, Stat5a, Stat5b, TCF3, TCF4, Klf12, SP3, Klf1, Klf4, KLF5, TBX4, TBX5, Pax2, MAFG::NFE2L1, EOMES, FOXD2, FOXO6, Pax2, MEIS1, MEIS2, MEIS3, SREBF2, or MGA, or a fragment thereof, is immobilized on the chip and a solution containing the oligonucleotide is contacted with the immobilized transcription factor

The invention also includes oligonucleotides targeting any transcript of a SNCA-mediated gene. For use as a therapeutic agent, such oligonucleotide may further comprise a pharmaceutically acceptable carrier. Optionally it may be chemically modified or formulated to enable transport into the brain across the blood-brain barrier. Preferred modifications include phosphorothioate oligonucleotide, a phosphoramidite oligonucleotide, a methylphosphonate oligonucleotide, a locked-nucleic acid-modified oligonucleotide, a peptide nucleic acid oligonucleotide, or combinations thereof. The invention includes methods for modulating expression of a SNCA-mediated gene in a cell, said method comprising contacting a cell with an oligonucleotide disclosed herein.

In another embodiment, the oligonucleotide is fused to a cell penetrating peptide.

In another embodiment, the oligonucleotide is a short-interfering RNA (siRNA). There are several methods for preparing siRNA, such as chemical synthesis, in vitro transcription, siRNA expression vectors, and PCR expression cassettes. Irrespective of which method one uses, the first step in designing a siRNA requires choosing the siRNA target site. Standard guidelines for choosing siRNA target sites available in the current literature. Using standard guidelines, approximately half of all siRNAs yield >50% reduction in target mRNA levels, thus providing compounds potentially useful in the treatment of PD, and of great interest for further investigation and secondary screening.

Antibodies and antigen binding fragments thereof. The term “antibody” as used to herein can include whole antibodies and refers, in one embodiment, to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. In certain naturally occurring IgG, IgD, and IgA antibodies, the heavy chain constant region is comprised of three domains, CH1, CH2 and CH3. In certain naturally occurring antibodies, each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FRI, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

Antibodies typically bind specifically to their cognate antigen with high affinity, reflected by a dissociation constant (K_{D}) of 10⁻⁷ to 10⁻¹¹ M or less. Any K_{D} greater than about 10⁻⁶ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸ M or less, even more preferably 5×10⁻⁹ M or less, and most preferably between 10⁻⁸ M and 10⁻¹⁰ M or less, but does not bind with high affinity to unrelated antigens.

Antibodies can also include, by way of example, both naturally occurring and non-naturally occurring antibodies; monoclonal and polyclonal antibodies; chimeric and humanized antibodies; fully-human antibodies and many variations known in the art.

The phrase “selectively binds to” refers to the ability of an antibody, antigen binding fragment or binding partner (antigen binding peptide) to preferentially bind to a SNCA-mediated gene expression product. Often the phrase “selectively binds” refers to the specific binding of antibody, fragment thereof, or binding partner to an antigen. The level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

Isolated antibodies of the invention can include serum containing such antibodies, or antibodies that have been purified to varying degrees. Whole antibodies of the invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), can also be employed in the invention.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies can be produced according to the methodology of Kohler and Milstein (Nature, 1975, 256, 495-497). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

The term “antigen-binding fragment” of an antibody, as used herein, refers to one or more fragments of an antibody that retain the ability to selectively bind to an antigen (e.g., the expression product of a SNCA-mediated gene). It has been shown that the antigen-binding function of an antibody can be performed by fragments of a full-length antibody. Examples of binding fragments encompassed within the term “antigen-binding fragment” of an antibody, e.g., an antibody directed to the expression product of a SNCA-mediated gene, include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) or (vii) a combination of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These and other potential constructs are described at Chan & Carter (2010) Nat. Rev. Immunol. 10:301. Those skilled in the art are also familiar with antigen-binding fragments such as minibodies, cys-diabodies and fibronectin binding domains, which are also included in the invention herein. These antigen binding fragments are obtained using conventional techniques known to those with skill in the art, and the fragments are screened for utility in the same manner as are intact antibodies. Antigen-binding fragments can be produced by recombinant DNA techniques, or by enzymatic or chemical cleavage of intact immunoglobulins.

A “bispecific” or “bifunctional antibody” is an artificial hybrid antibody having two different heavy/light chain pairs and two different binding sites. Bispecific antibodies can be produced by a variety of methods including fusion of hybridomas, or by linking of antigen-binding fragments. See, e.g., Songsivilai & Lachmann, Clin. Exp. Immunol. 79:315-321 (1990); Kostelny et al., J. Immunol. 148, 1547-1553 (1992). The invention includes a bispecific antibody for use in the treatment of PD when one or both of the binding sites are specific for the expression product of a SNCA-mediated gene. Based on the invention disclosed herein, an attractive avenue for therapeutic antibodies and antigen-binding fragments thereof include modifications to enhance transport from the blood to the brain across the blood-brain barrier (BBB).

Antibodies have demonstrated the capacity to cross the blood-brain barrier (“BBB”) on their own, often in cases of BBB defects (Prins and Scheltens (2013) Alzheimer's Research & Therapy 5:56; Doody et al. N Engl J Med 370:4). Antibody fragments able to do so have been isolated by phenotypic panning of a naive llama single-domain antibody phage display library. Single domain antibodies, also referred to as nanobodies, are derived from camelids, which make a unique subset of immunoglobulins consisting of heavy chain homodimers devoid of light chains. Their variable region (VHH) is the smallest antigen-binding single polypeptide chain naturally found in the antibody world. Selected antibodies FC5 and FC44 demonstrated significantly (p<0.01) enhanced transport (50-100-fold) across the BBB in a rat in vitro model compared to control VHHs.

An alternative to enhance BBB transport is to employ linker molecules that transport antibodies/fragments from the blood into the brain. For example, specific brain delivery is achieved by engineering bispecific antibodies in which a therapeutic “arm” is combined with a BBB-transcytosing arm. Such work is based on recognized BBB specific receptors and transporters. Many endogenous molecules in circulation are able to cross the BBB via specific receptors and transporters expressed on the luminal side of brain endothelial cells, a process known as receptor-mediated transcytosis. Antibodies generated against these receptors, e.g. transferrin receptor (TFRC), insulin receptor (INSR), low density lipoprotein receptor-related protein 1 (LRP1), Basigin (Ok Blood Group, BSG), Glucose Transporter Type 1 (SLC2A1) and solute carrier CD98hc (SLC3A2) have been shown to accumulate in the brain in vivo. These antibodies can be used as platform to deliver therapeutic antibodies across the BBB (Bispecific antibodies in which one half targets the transport system, and the other half is the therapeutic antibody). Bispecific antibodies against the transferrin receptor and BACE1 have been shown to traverse the blood-brain barrier and effectively reduce brain amyloid β levels.

The single-domain antibody FC5 has been shown to engage an active receptor mediated transport process by binding a putative α(2,3)-sialoglycoprotein receptor. Use of FC5 as the BBB-carrier arm in bispecific antibodies or antibody-drug conjugates offers an avenue to develop pharmacologically active biotherapeutics for CNS indications.

Another BBB transport moiety is the heavy-chain only antibodies identified in sharks. Antigen binding is mediated by a small and highly stable domain, known as VNAR. Antigen-specific VNAR molecules have been generated against a multitude of different targets via immunization, for instance VNAR that target the BBB transferrin receptor, as demonstrated by Ossianix Inc (Philadelphia, Pa.). This VNAR can be incorporated into a bispecific antibody, wherein the other binding moiety targets the expression product of a SNCA-mediated gene.

Diagnosis and prognosis of PD using SNCA-mediated genes: Another aspect of the present invention provides diagnostic assays for measuring levels of a SNCA-mediated gene, or its protein activity, in the context of a biological sample (e.g whole blood, blood plasma, blood serum, sputum, saliva, urine, lymph or cerebrospinal fluid) to thereby contribute to diagnosis of PD in a carrier of a SNCA genomic variant.

Tissues, cells or body fluids from subjects are collected and analyzed for expression levels of the SNCA-mediated gene or for expression levels of any of the genes listed in Table 4. There are numerous methods known to the skilled artisan to measure protein or mRNA expression levels in tissues, cells or body fluids. In a preferred embodiment, the tissue collected from subjects for expression analysis includes whole blood. In another preferred embodiment, fluids collected from subjects for expression analysis include whole blood, blood plasma, blood serum, sputum, saliva, urine, lymph or cerebrospinal fluid. In yet another preferred embodiment, cells collected from subjects include blood cells, buccal cells or skin fibroblasts.

An exemplary method for detecting the presence or absence of SNCA-mediated protein or nucleic acid in a biological sample involves obtaining a biological sample from a test subject and contacting the biological sample with a compound or an agent capable of detecting the protein or a nucleic acid (e.g., mRNA) that encodes the protein such that the presence of the protein or nucleic acid is detected in the biological sample. A preferred agent for detecting mRNA is a labeled nucleic acid probe capable of hybridizing to the mRNA. The nucleic acid probe can be, for example, a nucleic acid or a corresponding nucleic acid for SNCA-mediated gene such as an oligonucleotide of at least 15, 30, 50, 100, 250 or 500 nucleotides in length which is capable of specifically hybridizing under stringent conditions to the mRNA. Other suitable probes for use in the diagnostic assays of the invention are known to those skilled in the art.

A preferred agent for detecting protein expression is an antibody capable of binding to a protein expressed from an SNCA-mediated gene, preferably an antibody with a detectable label. Antibodies can be polyclonal, or more preferably, monoclonal. An intact antibody, or a fragment thereof (e.g., Fab or F(ab′)2) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

With respect to antibody-based detection techniques, one of skill in the art can raise antibodies against an appropriate immunogen of SNCA-mediated gene protein expression product using no more than routine experimentation. Conditions for using such antibodies in a diagnostic assay include incubating an antibody with a test sample under conditions that vary depending upon the tissue or cellular type. Incubation conditions can depend on the format employed in the assay, the detection methods employed, and the type and nature of the antibody used in the assay. One skilled in the art will recognize that any one of the commonly available immunological assay formats (such as radioimmunoassays, enzyme-linked immunosorbent assays, diffusion based Ouchterlony, or rocket immunofluorescent assays) can readily be adapted to employ an antibody of the invention. Examples of such assays can be found in Chard, “An Introduction to Radioimmunoassay and Related Techniques,” Elsevier Science Publishers, Amsterdam, The Netherlands (1986); Bullock et al., “Techniques in Immunocytochemistry,” Academic Press, Orlando, Fla. Vol. 1 (1982), Vol. 2 (1983), Vol. 3 (1985); Tijssen, “Practice and Theory of enzyme Immunoassays: Laboratory Techniques in Biochemistry and Molecular Biology,” is Elsevier Science Publishers, Amsterdam, The Netherlands (1985).

The diagnostic methods of the invention can be used to detect mRNA or protein of a SNCA-mediated gene in a biological sample in vitro as well as in vivo. For example, in vitro techniques for detection of mRNA include northern blot hybridizations and in situ hybridizations. In vitro techniques for detection of proteins include enzyme linked immunosorbent assays (ELISAs), Western blots, immunoprecipitations, immunofluorescence, or quantitative sequencing reactions. Protein or mRNA levels can also be measured in an assay designed to evaluate a panel of target genes, e.g., a microarray or multiplex sequencing reaction.

The invention also provides kits for detecting the presence of a SNCA-mediated gene transcript or its protein expression product in a biological sample. For example, the kit can comprise a labeled compound or agent capable of detecting such protein or mRNA in a biological sample; means for determining the amount of such protein or mRNA in the sample; and means for comparing the amount in the sample with a known standard. The compound or agent can be packaged in a suitable container. The kit can further comprise instructions for using the kit.

One of skill in the art would be capable of performing these well-established protocols for the methods of the invention. (See, for example, Ausubel, et al., “Current Protocols in Molecular Biology,” John Wiley & Sons, NY, N.Y. (1999)).

Companion diagnostic or selection of therapeutic agent: The diagnostic methods of the invention provide advantages in the selection of an appropriate therapeutic agent for treatment or prevention of PD. Given that a variety of therapeutic agents are employed in the treatment of symptoms of PD, and new therapeutics are anticipated in coming years, it is now possible to stratify patients who are responders or non-responders to such treatments on the basis of the activity or expression of the SNCA-mediated genes. Those of skill in the art can now perform clinical trials which correlate drug responsiveness with the results of one or more diagnostic assays for SNCA-mediated genes and their protein products.

At least two categories of such tests are immediately apparent. In one, a method for selecting a therapeutic agent for administration to a subject having, or at-risk of having PD, comprises measuring the activity of an SNCA-mediated gene in a biological sample from the subject, and selecting a therapeutic agent based on whether the subject demonstrates elevated activity of said gene in said sample. This example facilitates selection of a therapeutic agent of any type for the patient based on the diagnostic result.

In another method, a therapeutic agent which modulates the activity of an SNCA-mediated gene expression product may be recommended only if the subject demonstrates an elevated or decreased level of such gene expression product in the tissue sample. In this case the diagnostic test for an SNCA-mediated gene expression product is commonly known as a companion diagnostic. In such cases, if said subject does not demonstrate elevated levels of gene or protein activity, the treatment is different than if he/she does. In alternative cases, if said subject does not demonstrate decreased levels of gene expression or activity of a gene expression product, the treatment is different than if he/she does.

Experimental Validation of SNCA-Mediated Genes as Diagnostic and Therapeutic Targets in Human Neuronal Cells

Differentiated dopaminergic isogenic neurons readily available to the one skilled in the art served as the tissue source to isolate the starting genetic material necessary for the differential gene expression analysis using qPCR. To generate an isogenic A53T allelic variant nuclease-mediated SNP alteration of a healthy wild-type induced pluripotent stem (iPS) cell line was performed to introduce this site-specific mutation into the gene for SNCA. This genome-engineered iPS cell was then differentiated into human midbrain floorplate dopaminergic neurons to produce the A53T neurons. Hence, the two human dopaminergic neurons used in this analysis are homozygous for the SNCA A53T or the SNCA wild-type gene, respectively. Dopaminergic isogenic neurons with the homozygous SNCA wild-type or the homozygous SNCA genomic variant rs104893877 (A53T) genotype were compared for their expression of SNCA-mediated genes. The neurons carrying the homozygous SNCA wild-type are designated SNCA wt or SNCA wt/wt. The neurons carrying the homozygous SNCA genomic variant rs104893877 (A53T) are designated SNCA A53T or SNCA A53T/A53T. Both lines were cryopreserved, fully differentiated, >80% pure midbrain DA neurons that express the relevant midbrain dopaminergic neuron markers (e.g. Lmx1, FoxA2, and TH). Total RNA was extracted using the Direct-zol™ RNA MiniPrep Kit (Zymo Research, Irvine, Calif.; Cat. no. R2050) according to the manufacturer's instructions with optional on-column DNase treatment. Total RNA was extracted from 5.0×10⁶ A53T and 5.0×10⁶ WT neurons. RNA concentration and purity were determined using a Nanodrop ND-1000 spectrophotometer (Thermo Scientific). RNA integrity was measured using the Agilent 2100 Bioanalyzer (Agilent Technologies, Palo Alto, Calif.) and the RNA 6000 Nano Chip Kit according to the manufacturer's instructions. Subsequently, 3 g total RNA was used as template to synthesize cDNA with the High Capacity cDNA Reverse Transcription Kit (Applied Biosystems, Foster City, Calif.; Cat. no. 4368814). Detection of PCR products is enabled by the use of a fluorescent reporter molecule in the reaction that yields increased fluorescence with an increasing amount of product DNA. A method of detection was employed that involves the double-stranded DNA intercalating molecule SYBR Green® to determine gene expression levels of protein encoding genes located within 2 Mb of SNCA. Real time PCR was performed on the BioRad CFX384 Real Time System (BioRad, Hercules, Calif.) using the panel of genes of interest. Each reaction well contained 5 μL of PowerUp™ SYBR Green Master Mix (Applied Biosystems; Cat. no. A25742), cDNA equivalent to 13ng of total RNA and 250 nM each of forward and reverse amplification primers in a final reaction volume of 10 μL. Cycling conditions were as follows: 95° C. for 10 minutes for polymerase activation, followed by 40 cycles of 95° C. for 15 seconds and 60° C. for 1 minute. Glyceraldehyde-3-phosphate dehydrogenase (GAPDH), ubiquitin conjugating enzyme E2 D2 (UBE2D2), cytochrome c1 (CYC1) and ribosomal protein L13 (RPL13) were used as reference genes. Each quantitative qPCR experiment including amplification and data reduction performed in triplicates. Data analysis was performed using CFX Manager software from BioRad, version 3.1. The experimental Cq (cycle quantification) was calibrated against the endogenous control products. Quantification of nucleic acids was achieved using relative quantification analysis (Double delta Ct data analysis, ΔΔCt method). Relative quantification allows to determine fold-differences in expression of the target gene in the test cell line relative to the control cell line. First, a baseline was subtracted from the raw data based on the raw fluorescence values. Next, the threshold cycle (Ct) value was determined for each sample, which represents the number of cycles needed to reach a particular quantification threshold fluorescence signal level. Ct values were determined both for the genes being evaluated and for reference genes for normalization purposes. Average Ct values were determined for a gene of interest and the reference genes (designated as “Ref”) in the SNCA A53T neurons and SNCA wild-type control neurons. For further data analysis the SNCA A53T samples were compared to the SNCA wild-type control cell samples. For these paired comparison the following four values were generated: Avg. Ct Ref in SNCA A53T, Avg. Ct Ref in SNCA wild-type, Avg. Ct gene of interest in SNCA A53T, and Avg. Ct gene of interest in SNCA wild-type. The differences between Ct values of the gene of interest and reference genes (delta Ct values, short dCt) were calculated for the SNCA A53T and the control. Next, the difference between SNCA A53T and control was calculated to arrive at the Double Delta Ct Value (ddCt SNCA A53T-SNCA wild-type). Since the quantity of amplified product doubles in each cycle, the expression fold change between SNCA A53T and control (Relative Quantification or RQ (SNCA A53T/SNCA wild-type)) was computed with the following formula 2\hat{ }-ddCt.

TABLE 8
Relative expression levels of genes located within a 2 Mb range
of SNCA on human chromosome 4. Dopaminergic isogenic neurons with
the homozygous SNCA wild-type or the homozygous SNCA genomic
variant rs104893877 (A53T) genotype were compared. The neurons
carrying the homozygous SNCA wild-type are designated SNCA wt or
SNCA wt/wt. The neurons carrying the homozygous SNCA genomic
variant rs104893877 (A53T) are designated SNCA A53T or SNCA
A53T/A53T. Fold change means the relative expression level of
a gene in SNCA A53T neurons compared to SNCA wild-type neurons.
Negative values indicate downregulation in SNCA A53T/A53T versus
SNCA wt/wt neurons, positive values indicate upregulation.

| SNCA | SNCA A53T vs SNCA WT |
|---|---|
| mediated | mRNA expression |
| gene | (Fold change) |
| SPARCL1 | 3.222 |
| HERC6 | −2.292 |
| HERC5 | −2.126 |
| MMRN1 | 2.149 |

TABLE 9
Relative expression level of a gene located on human chromosome
12 differentially regulated in SNCA A53T/A53T neurons. Dopaminergic
isogenic neurons with the SNCA wt/wt or A53T/A53T genotype
were compared. Fold change means the relative expression
level of a gene in SNCA A53T neurons compared to SNCA wt
neurons. The positive value indicates upregulation in SNCA
A53T/A53T versus SNCA wt/wt neurons.

| | SNCA A53T/A53T vs |
|---|---|
| SNCA | SNCA WT/WT |
| mediated | mRNA expression |
| gene | (Fold change) |
| PDZRN4 | 2.721 |

Differentially regulated SNCA-mediated genes included SPARCL1, HERC6, HERC5, MMRN1 and PDZRN4. SPARCL1, MMRN1 and PDZRN4 were upregulated in SNCA A53T neurons compared to SNCA wt neurons. HERC5 and HERC6 were downregulated in SNCA A53T neurons compared to SNCA wt neurons.

PDZRN4 was downregulated in SNCA A53T neurons. The SNCA genomic variant rs104893877 (A53T) is linked to PD. The present invention discloses the dysregulation of the Notch pathway in PD. A decrease of PDZRN4 expression or the activity of a gene expression product thereof will improve at least one symptom of PD in a carrier of a SNCA genomic variant. PDZRN4, a member of the LNX E3 ubiquitin ligases can mediate the NUMB-dependent degradation of Notch. There are numerous assays known that measure the activity of E3 ubiquitin ligases on their substrates. Therefore, assays to measure the E3 ubiquitin ligase activity of PDZRN4 on its substrate Notch can be conceived by one skilled in the art.

A JASPAR analysis was performed and showed that the 5′ DNA region of MMRN1 contains several high scoring binding motifs for FOXO4 and FOXO6, MEIS1, 2, and 3, and Pax2 and one high scoring binding motif for NFIX. Therefore, the SNCA genomic variant rs104893877 (A53T) affects binding motifs for all these transcription factors.

The HERC5 promoter displays a binding site for TBX5. An increased score of the binding motif for this transcription factor is created by the SNCA genomic variant rs104893877 (A53T). A SREBF1/SREBP1 site was identified in the HERC5 promoter. The JASPAR analysis found several high scoring binding motifs for KLF4 and 5, NFIX and at least one high scoring binding motif for Pax2 within the HERC5 gene's 5′ DNA sequence. HERC5 expression is regulated through a transcription factor binding motif created by the SNCA genomic variant rs104893877 (A53T).

The JASPAR analysis defined high scoring binding sites for FOXD2, FOXO4 and 6, MEIS1 and 2, NFIX, SREBF2, TBX4/5, and MGA within the HERC6 gene's 5′ DNA sequence. Therefore, all these binding motives are affected by the SNCA genomic variant rs104893877 (A53T).

A JASPAR analysis was performed and showed that the 5′ DNA region of SPARCL1 contains several high scoring binding motifs for FOXD2, FOXO4, FOXO6. Four very high scoring binding motifs for MEIS1 and three very high scoring binding motifs for NFIX very identified as well. Therefore, all these binding motives are affected by the SNCA genomic variant rs104893877 (A53T). MEIS1 is strongly expressed in dopaminergic neurons of the substantia nigra. A de novo MEIS1 binding motif created by the A53T variant enhances MEIS1 controlled transcription of SPARCL1. On the other hand, NFIX can act either as transcription repressor or activator, depending of the gene and/or neighboring transcription factor binding sites and cellular context. Hence, the loss of the NFIX binding site in the A53T regions relieves the inhibitor effect by this transcription factor on the expression of SPARCL1.

The PDZRN4 5′region contains binding motifs for transcription factors MGA, NFIX, TBX1, TBX15, TBX4/5. PDZRN4 has been described as a target gene of NFIX in the hippocampus. A binding site for EOMES has been identified in the promoter of the PDZRN4 (CHEA Transcription Factor Targets dataset). A ZNF143 binding site was identified near the transcription start site of PDZRN4 (ENCODE Transcription Factor Target dataset). Transcription factors from the T-box factor family are involved in neuron differentiation and repression of pluripotency. MGA functions as a dual-specificity transcription factor that regulates the expression of both Max-network and T-box family target genes. Hence, de novo binding sites for these transcription factors in the A53T region allow increased expression of PDZRN4 while the loss of the NFIX binding site relieves the inhibitor effect by this transcription factor on the expression of PDZRN4.

Together the examples herein provide experimental validation of SNCA-mediated genes and expression products thereof as targets for diagnosis, prevention and treatment of Parkinson's disease in carriers of SNCA genomic variants.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

### REFERENCES

ⁱ Kett L R, Dauer W T. Leucine-rich repeat kinase 2 for beginners: six key questions. Cold Spring Harb Perspect Med. 2012 March; 2(3):a009407. doi: 10.1101/cshperspect.a009407. Review.
ⁱⁱ Li J Q, Tan L, Yu J T. The role of the SNCA gene in Parkinsonism. Mol Neurodegener. 2014 Nov. 12; 9:47. doi: 10.1186/1750-1326-9-47. Review.
ⁱⁱⁱ Soldner F, Stelzer Y, Shivalila C S, Abraham B J, Latourelle J C, Barrasa M I, Goldmann J, Myers R H, Young R A, Jaenisch R. Parkinson-associated risk variant in distal enhancer of α-synuclein modulates target gene expression. Nature. 2016 May 5; 533(7601):95-9. doi: 10.1038/nature17939. Epub 2016 Apr. 20.
^{iv} Li J Q, Tan L, Yu J T. The role of the SNCA gene in Parkinsonism. Mol Neurodegener. 2014 Nov. 12; 9:47. doi: 10.1186/1750-1326-9-47. Review.
^{v} Duda J E, Giasson B I, Mabon M E, Miller D C, Golbe L I, Lee V M, Trojanowski J Q. Concurrence of alpha-synuclein and tau brain pathology in the Contursi kindred. Acta Neuropathol. 2002 July; 104(1):7-11. Epub 2002 Apr. 27.
^{vi} Kiely A P, Ling H, Asi Y T, Kara E, Proukakis C, Schapira A H, Morris H R, Roberts H C, Lubbe S, Limousin P, Lewis P A, Lees A J, Quinn N, Hardy J, Love S, Revesz T, Houlden H, Holton J L. Distinct clinical and neuropathological features of G51D SNCA mutation cases compared with SNCA duplication and H50Q mutation. Mol Neurodegener. 2015 Aug. 27; 10:41. doi: 10.1186/s13024-015-0038-3.
^{vii} Obeso J A, Rodriguez-Oroz M C, Goetz C G, Marin C, Kordower J H, Rodriguez M, Hirsch E C, Farrer M, Schapira A H, Halliday G. Missing pieces in the Parkinson's disease puzzle. Nat Med. 2010 June; 16(6):653-61. doi: 10.1038/nm.2165. Epub 2010 May 23. Review.
^{viii} Rao S S, Huntley M H, Durand N C, Stamenova E K, Bochkov I D, Robinson J T, Sanborn A L, Machol I, Omer A D, Lander E S, Aiden E L. A 3D map of the human genome at kilobase resolution reveals principles of chromatin looping. Cell. 2014 Dec. 18; 159(7):1665-80. doi: 10.1016/j.cell.2014.11.021. Epub 2014 Dec. 11. Erratum in: Cell. 2015 Jul. 30; 162(3):687-8.
^{ix} Saura C A, Choi S Y, Beglopoulos V, Malkani S, Zhang D, Shankaranarayana Rao B S, Chattarji S, Kelleher R J 3rd, Kandel E R, Duff K, Kirkwood A, Shen J. Loss of presenilin function causes impairments of memory and synaptic plasticity followed by age-dependent neurodegeneration. Neuron. 2004 Apr. 8; 42(1):23-36.
^{x} Ables J L, Breunig J J, Eisch A J, Rakic P. Not(ch) just development: Notch signalling in the adult brain. Nat Rev Neurosci. 2011 May; 12(5):269-83. doi: 10.1038/nm3024.
^{xi} Hayashi Y, Nishimune H, Hozumi K, Saga Y, Harada A, Yuzaki M, Iwatsubo T, Kopan R, Tomita T. A novel non-canonical Notch signaling regulates expression of synaptic vesicle proteins in excitatory neurons. Sci Rep. 2016 Apr. 4; 6:23969. doi: 10.1038/srep23969.
^{xii} Perumalsamy L R, Nagala M, Sarin A. Notch-activated signaling cascade interacts with mitochondrial remodeling proteins to regulate cell survival. Proc Natl Acad Sci USA. 2010 Apr. 13; 107(15):6882-7. doi: 10.1073/pnas.0910060107. Epub 2010 Mar. 25.
^{xiii} Greene L A1, Levy O, Malagelada C. Akt as a victim, villain and potential hero in Parkinson's disease pathophysiology and treatment. Cell Mol Neurobiol. 2011 October; 31(7):969-78. doi: 10.1007/s10571-011-9671-8. Epub 2011 Mar. 10.
^{xiv} Alberi L, Liu S, Wang Y, Badie R, Smith-Hicks C, Wu J, Pierfelice T J, Abazyan B, Mattson M P, Kuhl D, Pletnikov M, Worley P F, Gaiano N. Activity-induced Notch signaling in neurons requires Arc/Arg3.1 and is essential for synaptic plasticity in hippocampal networks. Neuron. 2011 Feb. 10; 69(3):437-44. doi: 10.1016/j.neuron.2011.01.004.
^{xv} Byts N, Samoylenko A, Fasshauer T, Ivanisevic M, Hennighausen L, Ehrenreich H, Siren A L. Essential role for Stat5b in the neurotrophic but not in the neuroprotective effect of erythropoietin. Cell Death Differ. 2008 April; 15(4):783-92. doi: 10.1038/cdd.2008.1. Epub 2008 Feb. 8.
^{xvi} Takarada T, Kou M, Hida M, Fukumori R, Nakamura S, Kutsukake T, Kuramoto N, Hinoi E, Yoneda Y. Protective upregulation of activating transcription factor-3 against glutamate neurotoxicity in neuronal cells under ischemia. J Neurosci Res. 2016 May; 94(5):378-88. doi: 10.1002/jnr.23723. Epub 2016 Feb. 22.
^{xvii} Mason M J, Plath K, Zhou Q. Identification of context-dependent motifs by contrasting ChIP binding data. Bioinformatics. 2010 Nov. 15; 26(22):2826-32. doi: 10.1093/bioinformatics/btq546. Epub 2010 Sep. 23.
^{xviii} Moore D L, Apara A, Goldberg J L. Kruppel-like transcription factors in the nervous system: novel players in neurite outgrowth and axon regeneration. Mol Cell Neurosci. 2011 August; 47(4):233-43. doi: 10.1016/i.mcn.2011.05.005. Epub 2011 May 24.
^{xix} Hurlin P J, Steingrimsson E, Copeland N G, Jenkins N A, Eisenman R N. Mga, a dual-specificity transcription factor that interacts with Max and contains a T-domain DNA-binding motif EMBO J. 1999 Dec. 15; 18(24):7019-28.
^{xx} Soukup S F, Vanhauwaert R, Verstreken P. Parkinson's disease: convergence on synaptic homeostasis. EMBO J. 2018; 37(18):e98960. doi:10.15252/embj.201898960.
^{xxi} Rouillard A D, Gundersen G W, Fernandez N F, Wang Z, Monteiro C D, McDermott M G, Ma'ayan A. The harmonizome: a collection of processed datasets gathered to serve and mine knowledge about genes and proteins. Database (Oxford). 2016 Jul. 3; 2016. pii: baw100.
^{xxii} Hashimoto N, Sato T, Yajima T, Fujita M, Sato A, Shimizu Y, Shimada Y, Shoji N, Sasano T, Ichikawa H SPARCL1-containing neurons in the human brainstem and sensory ganglion. Somatosens Mot Res. 2016 June; 33(2):112-7. doi: 10.1080/08990220.2016.1197115. Epub 2016 Jun. 29.
^{xxiii} Sara Y, Biederer T, Atasoy D, Chubykin A, Mozhayeva M G, Sudhof T C, Kavalali E T. Selective capability of SynCAM and neuroligin for functional synapse assembly. J Neurosci. 2005 Jan. 5; 25(1):260-70
^{xxiv} Lin Y H, Chang H M, Chang F P, Shen C R, Liu C L, Mao W Y, Lin C C, Lee H S, Shen C N. Protoporphyrin I X accumulation disrupts mitochondrial dynamics and function in ABCG2-deficient hepatocytes. FEBS Lett. 2013 Oct. 1; 587(19):3202-9. doi: 10.1016/j.febslet.2013.08.011. Epub 2013 Aug. 14.
^{xxv} Lu G, Ren S, Korge P, Choi J, Dong Y, Weiss J, Koehler C, Chen J N, Wang Y. A novel mitochondrial matrix serine/threonine protein phosphatase regulates the mitochondria permeability transition pore and is essential for cellular survival and development. Genes Dev. 2007 Apr. 1; 21(7):784-96. Epub 2007 Mar. 20.
^{xxvi} Gautier C A, Giaime E, Caballero E, Núñez L, Song Z, Chan D, Villalobos C, Shen J. Regulation of mitochondrial permeability transition pore by PINK1. Mol Neurodegener. 2012 May 25; 7:22. doi: 10.1186/1750-1326-7-22.
^{xxvii} Aksoy I, Giudice V, Delahaye E, Wianny F, Aubry M, Mure M, Chen J, Jauch R, Bogu G K, Nolden T, Himmelbauer H, Xavier Doss M, Sachinidis A, Schulz H, Hummel O, Martinelli P, Hubner N, Stanton L W, Real F X, Bourillot P Y, Savatier P. Klf4 and Klf5 differentially inhibit mesoderm and endoderm differentiation in embryonic stem cells. Nat Commun. 2014 Apr. 28; 5:3719. doi: 10.1038/ncomms4719.
^{xxviii} Wong J J, Pung Y F, Sze N S, Chin K C. HERC5 is an IFN-induced HECT-type E3 protein ligase that mediates type I IFN-induced ISGylation of protein targets. Proc Natl Acad Sci USA. 2006 Jul. 11; 103(28):10735-40. Epub 2006 Jun. 30.
^{xxix} Im E, Yoo L, Hyun M, Shin W H, Chung K C. Covalent ISG15 conjugation positively regulates the ubiquitin E3 ligase activity of parkin. Open Biol. 2016 August; 6(8). pii: 160193. doi: 10.1098/rsob.160193.
^{xxx} Arimoto K, Hishiki T, Kiyonari H, Abe T, Cheng C, Yan M, Fan J B, Futakuchi M, Tsuda H, Murakami Y, Suzuki H, Zhang D E, Shimotohno K. Murine Herc6 Plays a Critical Role in Protein ISGylation In Vivo and Has an ISGylation-Independent Function in Seminal Vesicles. J Interferon Cytokine Res. 2015 May; 35(5):351-8. doi: 10.1089/jir.2014.0113. Epub 2014 Nov. 19.

What is claimed is:

1. A method for detecting a change in the expression of at least one SNCA-mediated gene comprising isolating total RNA from a tissue or cell sample obtained from a carrier of a SNCA genomic variant suspected of having Parkinson's disease and synthesizing and amplifying SNCA-mediated gene cDNA from said tissue or cell sample and identifying a change in the expression of at least one SNCA-mediated gene, wherein the change in the expression of the at least one SNCA-mediated gene comprises a change in the amount of mRNA encoded by the at least one SNCA-mediated gene compared to the amount in total RNA isolated from a same tissue or cell sample from a healthy subject.

2. The method of claim 1, wherein the change in the expression of the at least one SNCA-mediated gene is detected in total RNA isolated from tissue or cell samples obtained from carriers of a SNCA genomic variant with PD.

3. The method of claim 1, wherein the at least one SNCA-mediated gene is located within about 2 Mb of the SNCA gene.

4. The method of claim 1, wherein the at least one SNCA-mediated gene comprises at least one of KLHL8, PTPN13, HSDT7B13, HSDT7B11, NUDT9, SPARCL1, DMP1, IBSP, MEPE, SPP1, PKD2, ABCG2, PPM1K, HERC6, HERC5, PIGY, PYURF, HERC3, NAP1L5, FAM13A, TIGD2, GPRIN3, SNCA-AS1, MMRN1, CCSER1, GRID2 and PDZRN4.

5. The method of claim 1, wherein the at least one SNCA-mediated gene comprises at least two of KLHL8, PTPN13, HSD17B13, HSD17B11, NUDT9, SPARCL1, DMP1, IBSP, MEPE, SPP1, PKD2, ABCG2, PPM1K, HERC6, HERC5, PIGY, PYURF, HERC3, NAP1L5, FAM13A, TIGD2, GPRIN3, SNCA-AS1, MMRN1, CCSER1, GRID2 and PDZRN4.

6. The method of claim 1, wherein the at least one SNCA-mediated gene comprise at least three of KLHL8, PTPN13, HSD17B13, HSD17B11, NUDT9, SPARCL1, DMP1, IBSP, MEPE, SPP1, PKD2, ABCG2, PPM1K, HERC6, HERC5, PIGY, PYURF, HERC3, NAP1L5, FAM13A, TIGD2, GPRIN3, SNCA-AS1, MMRN1, CCSER1, GRID2 and PDZRN4.

7. The method of claim 1, wherein the at least one SNCA-mediated gene comprises at least one of ABCG2, PPM1K, HERC3, HERC5, HERC6, SPARCL1, MMRN1 and PDZRN4.

8. The method of claim 1, wherein the at least one SNCA-mediated gene comprises at least one of HERC5, HERC6, SPARCL1, MMRN1 and PDZRN4.

9. The method of claim 1, wherein the expression of at least one SNCA-mediated gene is greater than its expression in total RNA isolated from the same tissue or cell sample obtained from a healthy subject.

10. The method of claim 9, wherein the at least one SNCA-mediated gene comprises at least one of SPARCL1, MMRN1 and PDZRN4.

11. The method of claim 1, wherein the expression of the least one SNCA-mediated gene is less than the expression of the SNCA-mediated gene in total RNA isolated from the same tissue or cell sample obtained from a healthy subject.

12. The method of claim 11, wherein the at least one SNCA-mediated gene is HERC5 or HERC6.

13. The method of claim 1, wherein the tissue or cell sample is whole blood, blood plasma, blood serum, sputum, saliva, urine, lymph or cerebrospinal fluid, human homozygous or heterozygous SNCA variant neurons, buccal cells, or skin fibroblasts.

14. A method for improving at least one symptom in a carrier of a SNCA genomic variant with PD comprising administering a molecule to said carrier exhibiting a change in the expression of, or the activity of a gene expression product of at least one SNCA-mediated gene; said molecule modulating a change in the expression of, or an activity of a gene expression product by, at least one SNCA-mediated gene, and said modulation correlating with the improvement of at least one symptom.

15. The method of claim 14, wherein the SNCA-mediated gene is at least one of KLHL8, PTPN13, HSD17B13, HSD17B11, NUDT9, SPARCL1, DMP1, IBSP, MEPE, SPP1, PKD2, ABCG2, PPM1K, HERC6, HERC5, PIGY, PYURF, HERC3, NAP1L5, FAM13A, TIGD2, GPR1N3, SNCA-AS1, MMRN1, CCSER1, GRID2 and PDZRN4.

16. The method of claim 14, wherein the change is an increase in the expression of, or an activity of a gene expression product encoded by, the at least one SNCA-mediated gene.

17. The method of claim 16, wherein the expression of, or an activity of a gene expression product, of the at least one SNCA-mediated gene is modulated by administering to said carrier a molecule, said molecule causing a decrease in the expression of, or an activity of a gene expression product of, a SNCA-mediated gene, and said decrease in expression or activity correlating with an improvement of at least one symptom.

18. The method of claim 17 wherein said molecule comprises at least one of a small molecule, an oligonucleotide, a peptide, a polypeptide, or a protein.

19. The method of claim 14, wherein the change is a decrease in the expression of, or an activity of a gene expression product encoded by, the at least one SNCA-mediated gene.

20. The method of claim 19, wherein the expression of, or an activity of a gene expression product, of the at least one SNCA-mediated gene is modulated by administering to said carrier a molecule, said molecule causing an increase in the expression of, or an activity of a gene expression product of, a SNCA-mediated gene, and said increase in expression or activity correlating with an improvement of at least one symptom.

21. The method of claim 20 wherein said molecule comprises at least one of a small molecule, an oligonucleotide, a peptide, a polypeptide, or a protein.

22. A cell-based assay for detecting a change in the expression of, or an activity of a gene expression product encoded by, an SNCA-mediated gene.

23. A method for detecting a change in the expression of, or an activity of a gene expression product encoded by, a SNCA-mediated gene comprising:
measuring the expression of, or the activity of a gene expression product encoded by, a SNCA-mediated gene in cells having a SNCA genomic variant and
detecting a change in the expression of or an activity in response to a molecule,
wherein the change detected comprises a change in the synthesis of a gene expression product, an activity of the gene expression product or the expression of an mRNA encoded by the SNCA-mediated gene.

24. The method of claim 23, wherein the expression of, or an activity of a gene expression product encoded by, the SNCA-mediated gene increases in response to a molecule.

25. The method of claim 24, wherein the SNCA-mediated gene is at least one of HERC5 or HERC6.

26. The method of claim 23, wherein the expression of, or the activity of a gene expression product encoded by, the SNCA-mediated gene decreases in response to a molecule.

27. The method of claim 26, wherein the SNCA-mediated gene is at least one of SPARCL1, MMRN1 and PDZRN4.

28. The method of claim 24 or 26, wherein the change in expression of, or the activity of a gene expression product encoded by, the SNCA-mediated gene is detected by a change in the amount of phospho-Ser473-Akt or Notch.

29. The method of claim 24 or 26, wherein the molecule is potentially useful for treating or preventing the progression of Parkinson's disease (PD) in a carrier of a SNCA genomic variant.

30. The method according to claim 23 wherein the cells are human cells

31. The method according to claim 23 wherein the cells are neuronal cells, neuronal progenitor cells, differentiated neurons or oligodendrocytes.

32. The method according to claim 23 wherein the molecule is a small molecule, an oligonucleotide, a peptide, a polypeptide, or a protein.

33. A kit for detecting a change in the expression of at least one SNCA-mediated gene in total RNA isolated a sample obtained from a carrier of a SNCA genomic variant suspected of having Parkinson's disease comprising an agent for detecting mRNA encoded by at least one SNCA-mediated gene comprising at least one of KLHL8, PTPN13, HSD17B13, HSD17B11, NUDT9, SPARCL1, DMP1, IBSP, MEPE, SPP1, PKD2, ABCG2, PPM1K, HERC6, HERC5, PIGY, PYURF, HERC3, NAP1L5, FAM13A, TIGD2, GPR1N3, SNCA-AS1, MMRN1, CCSER1, GRID2 and PDZRN4.

34. The method according to claim 1 or 23 wherein the assay is a cell-based assay for detecting the change in the expression of, or an activity of a gene expression product encoded by, a SNCA-mediated gene.
